SEQUENCE LISTING (43 sequences)

SEQ ID NO: 1 - DNA, 19 bp, Homo sapiens
  misc_feature (1)..(19): Target sequence in TRAC gene
cagggttctggatatctgt

SEQ ID NO: 2 - DNA, 2119 bp, Artificial Sequence
  Synthetic anti-CD38 CAR Construct for insertion into TRAC locus
  misc_feature: JeT promoter, DNA sequence encoding CD8a leader peptide, anti-CD38 CAR (single chain variable fragment (scFv) specific for human CD38), CD28 hinge-transmembrane- intracellular regions and CD3 zeta intracellular domain
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactcccaggtagagcagaaattgatctctgaggaagacctgcaggtccagttggtcgaaagtggcggcggattggtgaaaccaggcggatctttgaggcttagttgcgcggcttccggatttacgttcagtgatgactacatgagctggataaggcaagcacctggtaagggcctggaatgggtcgcaagtgtgtctaatggaaggcccactacctactatgctgattccgtccgcggacgctttactatttcaagagataatgctaagaatagtctgtacctgcagatgaacagtctgcgcgcggaagataccgcagtatattactgtgcacgagaggattggggtggggagttcacggattggggcaggggaactcttgtaacggtgtctagcggaggaggtgggtcaggtggaggtggcagtggaggtggaggctctcaggccggcttgacccaaccgccatctgcgtcaggaacatcaggccagagggtgactatcagttgttctggcagttcatccaatattgggatcaatttcgtgtactggtatcagcacctgccaggtaccgcgccgaagctgctgatctataagaataatcaacgcccatcaggcgttccagataggttcagtgggagcaagtccggaaactccgcgtcactcgcgatctcaggtctgcggtctgaggatgaagctgattattactgcgcggcgtgggatgattctctgtcaggctacgtattcggttcagggactaaggtaactgtgttggcgaaaccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacggccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 3 - DNA, 195 bp, Artificial Sequence
  Synthetic Jet promoter
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcagttgaca

SEQ ID NO: 4 - DNA, 3429 bp, Artificial Sequence
  Synthetic anti-CD38A2 CAR cassette with homology arms
ggcaccatattcattttgcaggtgaaattcctgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactcccaggtagagcagaaattgatctctgaggaagacctgcaggtccagttggtcgaaagtggcggcggattggtgaaaccaggcggatctttgaggcttagttgcgcggcttccggatttacgttcagtgatgactacatgagctggataaggcaagcacctggtaagggcctggaatgggtcgcaagtgtgtctaatggaaggcccactacctactatgctgattccgtccgcggacgctttactatttcaagagataatgctaagaatagtctgtacctgcagatgaacagtctgcgcgcggaagataccgcagtatattactgtgcacgagaggattggggtggggagttcacggattggggcaggggaactcttgtaacggtgtctagcggaggaggtgggtcaggtggaggtggcagtggaggtggaggctctcaggccggcttgacccaaccgccatctgcgtcaggaacatcaggccagagggtgactatcagttgttctggcagttcatccaatattgggatcaatttcgtgtactggtatcagcacctgccaggtaccgcgccgaagctgctgatctataagaataatcaacgcccatcaggcgttccagataggttcagtgggagcaagtccggaaactccgcgtcactcgcgatctcaggtctgcggtctgaggatgaagctgattattactgcgcggcgtgggatgattctctgtcaggctacgtattcggttcagggactaaggtaactgtgttggcgaaaccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacggccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatcttt

SEQ ID NO: 5 - DNA, 20 bp, Artificial Sequence
  Synthetic primer for sequencing clones with anti-CD38 CAR-TRAC homology arms insert in pAAV-MCS vector
cttaggctgggcattagcag

SEQ ID NO: 6 - DNA, 20 bp, Artificial Sequence
  Synthetic primer for sequencing clones with anti-CD38 CAR-TRAC homology arms insert in pAAV-MCS vector
catggaatggtcatgggtct

SEQ ID NO: 7 - DNA, 20 bp, Artificial Sequence
  Synthetic primer for sequencing clones with anti-CD38 CAR-TRAC homology arms insert in pAAV-MCS vector
ggctacgtattcggttcagg

SEQ ID NO: 8 - DNA, 20 bp, Artificial Sequence
  Synthetic Forward primer for generating donor DNA PCR fragment from pAAV anti-CD38 CAR-TRAC construct (660 & 650 HAs)
  modified_base (1)..(2): Phosphorothioate linkage
  modified_base (2)..(3): Phosphorothioate linkage
  modified_base (2)..(3): 2′-O-methylated deoxyguanosine
  modified_base (3)..(4): Phosphorothioate linkage
  modified_base (4)..(4): 2′-O-methylated deoxyadenosine
tggagctagggcaccatatt

SEQ ID NO: 9 - DNA, 20 bp, Artificial Sequence
  Synthetic Reverse primer for generating donor DNA PCR fragment from pAAV anti-CD38 CAR-TRAC construct (660 & 650 HAs), the 5-most nucleoside (C) has a 5 phosphate
caacttggagaaggggctta

SEQ ID NO: 10 - DNA, 19 bp, Artificial Sequence
  Synthetic PCR forward primer with homology to TRAC locus for verifying site-specific insertion of anti-CD38 CAR (upstream junction)
ctgctttctgagggtgaag

SEQ ID NO: 11 - DNA, 20 bp, Artificial Sequence
  Synthetic PCR reverse primer with homology to CAR construct for verifying site-specific insertion of anti-CD38 CAR (upstream junction)
ctttcgaccaactggacctg

SEQ ID NO: 12 - DNA, 20 bp, Artificial Sequence
  Synthetic PCR forward primer with homology to CAR locus for verifying site-specific insertion of anti-CD38 CAR (downstream junction)
cgttctgggtactcgtggtt

SEQ ID NO: 13 - DNA, 20 bp, Artificial Sequence
  Synthetic PCR reverse primer with homology to TRAC locus (downstream junction)
gagagcccttccctgacttt

SEQ ID NO: 14 - DNA, 20 bp, Artificial Sequence
  Synthetic Forward primer for generating donor fragment with 300 nt HAs
  modified_base (1)..(2): Phosphorothioate linkage
  modified_base (2)..(3): Phosphorothioate linkage
  modified_base (2)..(2): 2-O-methylated deoxycytosine
  modified_base (3)..(4): Phosphorothioate linkage
  modified_base (3)..(3): 2′-O-methylated deoxyadenosine
  modified_base (5)..(5): 2′-O-methylated deoxyguanosine
ccatgcctgcctttactctg

SEQ ID NO: 15 - DNA, 20 bp, Artificial Sequence
  Synthetic Reverse primer for generating donor fragment with 300 nt HAs, the 5-most nucleoside (T) has a 5 phosphate
tcctgaagcaaggaaacagc

SEQ ID NO: 16 - DNA, 375 bp, Homo sapiens
  misc_feature (1)..(375): 5 homology arm, exon 1 TRAC gene
ccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 17 - DNA, 321 bp, Homo sapiens
  misc_feature (1)..(321): 3 homology arm, exon 1 TRAC gene
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaggacaccttcttccccagcccaggtaagggcagctttggtgccttcgcaggctgtttccttgcttcagga

SEQ ID NO: 18 - DNA, 20 bp, Artificial Sequence
  Synthetic Forward primer for generating donor fragment with 150 nt HAs
  modified_base (1)..(2): Phosphorothioate linkage
  modified_base (3)..(4): Phosphorothioate linkage
  modified_base (3)..(3): 2-O-methylated deoxycytosine
  modified_base (4)..(5): Phosphorothioate linkage
  modified_base (4)..(4): 2′-O-methylated deoxyadenosine
  modified_base (5)..(5): 2-O-methylated deoxycytosine
atcacgagcagctggtttct

SEQ ID NO: 19 - DNA, 24 bp, Artificial Sequence
  Synthetic Reverse primer for generating donor fragment with 150 nt HAs, the 5-most nucleoside (G) has a 5 phosphate
gacctcatgtctagcacagttttg

SEQ ID NO: 20 - DNA, 171 bp, Homo sapiens
  misc_feature (1)..(171): 5 171 nt homology arm, exon 1 TRAC gene
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaca

SEQ ID NO: 21 - DNA, 161 bp, Homo sapiens
  misc_feature (1)..(161): 3 161 nt homology arm, exon 1 TRAC gene
gatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 22 - DNA, 2080 bp, Artificial Sequence
  Synthetic anti-CD19 CAR cassette including JeT promoter, intron, anti-CD19 CAR, SV40 sequence
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactccgatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcgctaagccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 23 - DNA, 2083 bp, Artificial Sequence
  Synthetic anti-BCMA CAR construct including JeT promoter, intron, anti-BCMA CAR construct, SV40 sequence
  misc_feature (1057)..(1057): n is a, c, g, or t
  misc_feature (1098)..(1098): n is a, c, g, or t
  misc_feature (1120)..(1120): n is a, c, g, or t
gaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggtcctgggtgttcctgttctttctgtccgtgaccaccggtgtccactctcaggtgcagctggtggagtctgggggaggcttggtaaagcctggggggtcccttagactctcctgtgcagcctctggattcacttccagtaccgcctggatgagctgggtccgccaggctccagggaaggggctggagtgggttggccgtattaaaagcaaaagtgatggtgggacaacagactacgctgcacccgtgaaaggcagattcaccatctcaagagatgattcaaaaaacacgctgtttctgcaaatgaacagcctgaaaaccgaggacacagccgtgtattactgtgccaagggaggcgggacctacggctactggggccagggaaccctggtcaccgtctcctccggcggcggcggcagcggtggcggtggctcaggtggtggtggttcttcctatgtgctgactcagcctgcctccgtgtctgggtctcctggacagtcagtcaccatctcctgcactggaaccagcagtgatggtggtggtcacacctatgtctcctggtaccaacagcacccaggcaaagcccccaaactcatgatttatgatgtcagtaatcggccctcatgggtttctaatcgcttctctggctccaagtctggcaacacggcctccctgaccatctctgggctccaggctgangacgaggctgattattactgcggctcatatacaagcagcgnctcttatgtcttcggaactggnaccaagctgaccgtcctggctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 24 - DNA, 183 bp, Homo sapiens
  misc_feature (1)..(183): 5 homology arm, TRAC gene exon 3, 183 nt
tatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtc

SEQ ID NO: 25 - DNA, 140 bp, Homo sapiens
  misc_feature (1)..(140): 3 homology arm, TRAC gene exon 3
agtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaa

SEQ ID NO: 26 - DNA, 20 bp, Homo sapiens
  misc_feature (1)..(20): Exon 3 target sequence (guide sequence)
ttcggaacccaatcactgac

SEQ ID NO: 27 - DNA, 2442 bp, Artificial Sequence
  Synthetic Entire donor DNA anti-CD38 CAR plus exon 3 HAs on both ends
tatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactcccaggtagagcagaaattgatctctgaggaagacctgcaggtccagttggtcgaaagtggcggcggattggtgaaaccaggcggatctttgaggcttagttgcgcggcttccggatttacgttcagtgatgactacatgagctggataaggcaagcacctggtaagggcctggaatgggtcgcaagtgtgtctaatggaaggcccactacctactatgctgattccgtccgcggacgctttactatttcaagagataatgctaagaatagtctgtacctgcagatgaacagtctgcgcgcggaagataccgcagtatattactgtgcacgagaggattggggtggggagttcacggattggggcaggggaactcttgtaacggtgtctagcggaggaggtgggtcaggtggaggtggcagtggaggtggaggctctcaggccggcttgacccaaccgccatctgcgtcaggaacatcaggccagagggtgactatcagttgttctggcagttcatccaatattgggatcaatttcgtgtactggtatcagcacctgccaggtaccgcgccgaagctgctgatctataagaataatcaacgcccatcaggcgttccagataggttcagtgggagcaagtccggaaactccgcgtcactcgcgatctcaggtctgcggtctgaggatgaagctgattattactgcgcggcgtgggatgattctctgtcaggctacgtattcggttcagggactaaggtaactgtgttggcgaaaccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacggccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttaagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaa

SEQ ID NO: 28 - DNA, 21 bp, Artificial Sequence
  Synthetic Forward primer for generating donor fragment of SEQ ID NO27
  modified_base (1)..(2): Phosphorothioate linkage
  modified_base (2)..(3): Phosphorothioate linkage
  modified_base (2)..(2): 2′-O-methylated deoxyadenosine
  modified_base (3)..(4): Phosphorothioate linkage
  modified_base (4)..(4): 2′-O-methylated deoxyguanosine
  modified_base (5)..(5): 2-O-methylated deoxycytosine
tatgccacagaagctgcaagg

SEQ ID NO: 29 - DNA, 20 bp, Artificial Sequence
  Synthetic Reverse primer for generating donor fragment of SEQ ID NO 27
  misc_feature (1)..(1): 5-most nucleoside (T) has a 5 phosphate
ttaggatgcacccagagacc

SEQ ID NO: 30 - DNA, 326 bp, Homo sapiens
  misc_feature (1)..(326): PD-1 locus 5 HA
ctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgccctggccagtcgtctgggcggtgctacaactgggctggcggccaggatggttctta

SEQ ID NO: 31 - DNA, 380 bp, Homo sapiens
  misc_feature (1)..(380): PD-1 locus 3 HA
ggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcagg

SEQ ID NO: 32 - DNA, 20 bp, Homo sapiens
  misc_feature (1)..(20): PD-1 target site
ggccaggatggttcttaggt

SEQ ID NO: 33 - DNA, 2825 bp, Artificial Sequence
  Synthetic Donor DNA fragment having CD38 cassette (SEQ ID NO2) flanked by PD-1 HAs
ctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatgcagatcccacaggcgccctggccagtcgtctgggcggtgctacaactgggctggcggccaggatggttcttagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactcccaggtagagcagaaattgatctctgaggaagacctgcaggtccagttggtcgaaagtggcggcggattggtgaaaccaggcggatctttgaggcttagttgcgcggcttccggatttacgttcagtgatgactacatgagctggataaggcaagcacctggtaagggcctggaatgggtcgcaagtgtgtctaatggaaggcccactacctactatgctgattccgtccgcggacgctttactatttcaagagataatgctaagaatagtctgtacctgcagatgaacagtctgcgcgcggaagataccgcagtatattactgtgcacgagaggattggggtggggagttcacggattggggcaggggaactcttgtaacggtgtctagcggaggaggtgggtcaggtggaggtggcagtggaggtggaggctctcaggccggcttgacccaaccgccatctgcgtcaggaacatcaggccagagggtgactatcagttgttctggcagttcatccaatattgggatcaatttcgtgtactggtatcagcacctgccaggtaccgcgccgaagctgctgatctataagaataatcaacgcccatcaggcgttccagataggttcagtgggagcaagtccggaaactccgcgtcactcgcgatctcaggtctgcggtctgaggatgaagctgattattactgcgcggcgtgggatgattctctgtcaggctacgtattcggttcagggactaaggtaactgtgttggcgaaaccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacggccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcagg

SEQ ID NO: 34 - DNA, 20 bp, Artificial Sequence
  Synthetic Forward primer for generating donor fragment of SEQ ID NO33
  misc_feature: 5-most nucleoside (C) has a 5 phosphate
ctccccatctcctctgtctc

SEQ ID NO: 35 - DNA, 21 bp, Artificial Sequence
  Synthetic Reverse primer for generating donor fragment of SEQ ID NO33
  modified_base (1)..(2): Phosphorothioate linkage
  modified_base (1)..(1): 2-O-methylated deoxycytosine
  modified_base (2)..(3): Phosphorothioate linkage
  modified_base (2)..(2): 2-O-methylated deoxycytosine
  modified_base (3)..(4): Phosphorothioate linkage
  modified_base (4)..(4): 2′-O-methylated deoxyguanosine
cctggacccgtcattctacag

SEQ ID NO: 36 - DNA, 20 bp, Homo sapiens
  misc_feature (1)..(20): Forward primer for generating donor DNA PCR fragment from pAAV anti-CD38 CAR-TRAC construct (660 & 650 HAs)
tggagctagggcaccatatt

SEQ ID NO: 37 - DNA, 2415 bp, Artificial Sequence
  Synthetic anti-BCMA CAR construct donor fragment sequence, including JeT promoter, intron, anti-BCMA CAR construct, SV40 sequence, with 5 and 3 TRAC gene exon 1 homology arms
  misc_feature (1228)..(1228): n is a, c, g, or t
  misc_feature (1269)..(1269): n is a, c, g, or t
  misc_feature (1291)..(1291): n is a, c, g, or t
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggagtggtcctgggtgttcctgttctttctgtccgtgaccaccggtgtccactctcaggtgcagctggtggagtctgggggaggcttggtaaagcctggggggtcccttagactctcctgtgcagcctctggattcacttccagtaccgcctggatgagctgggtccgccaggctccagggaaggggctggagtgggttggccgtattaaaagcaaaagtgatggtgggacaacagactacgctgcacccgtgaaaggcagattcaccatctcaagagatgattcaaaaaacacgctgtttctgcaaatgaacagcctgaaaaccgaggacacagccgtgtattactgtgccaagggaggcgggacctacggctactggggccagggaaccctggtcaccgtctcctccggcggcggcggcagcggtggcggtggctcaggtggtggtggttcttcctatgtgctgactcagcctgcctccgtgtctgggtctcctggacagtcagtcaccatctcctgcactggaaccagcagtgatggtggtggtcacacctatgtctcctggtaccaacagcacccaggcaaagcccccaaactcatgatttatgatgtcagtaatcggccctcatgggtttctaatcgcttctctggctccaagtctggcaacacggcctccctgaccatctctgggctccaggctgangacgaggctgattattactgcggctcatatacaagcagcgnctcttatgtcttcggaactggnaccaagctgaccgtcctggctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 38 - DNA, 2217 bp, Artificial Sequence
  Synthetic anti-CD19 CAR cassette including JeT promoter, intron,  anti-CD19 CAR, SV40 sequence flanked by 5 and 3 TRAC gene exon 1 homology arms of SEQ ID NO20 and SEQ ID NO21
atcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggcctcgaggccgccaccatggaatggtcatgggtctttctcttttttctcagcgtgaccaccggagtccactccgatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcgctaagccgaccacgacaccggctccaagacctccgacgccagctccaacgatagcgtcacagccattgtctctccgccctgaagcctgccggcccgctgcgggcggcgcggttcatacccggggattggactttgcccccagaaagatagaggtgatgtaccctcccccctacttggacaacgaaaagtctaatggcactatcattcacgtaaagggcaaacacctttgtccaagtcctttgttcccaggcccatctaagccgttctgggtactcgtggttgtggggggcgtgctcgcttgttactcactgctggtgacggtggcctttattattttctgggttcgatctaagcgaagccgcttgttgcattctgactacatgaatatgacgccaagacggccagggccaacaagaaagcattaccaaccgtacgcccccccgcgagacttcgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtc

SEQ ID NO: 39 - DNA, 1001 bp, Artificial Sequence
  Synthetic Sequenced PCR product of 5 end of donor DNA plus adjacent TRAC gene exon 1 genomic sequence that included portion of anti-CD38 CAR construct and 660 nt homology arm
  misc_feature (1)..(15): n is a, c, g, or t
  misc_feature (18)..(19): n is a, c, g, or t
  misc_feature (790)..(792): n is a, c, g, or t
  misc_feature (925)..(925): n is a, c, g, or t
  misc_feature (931)..(931): n is a, c, g, or t
  misc_feature (933)..(934): n is a, c, g, or t
  misc_feature (1001)..(1001): n is a, c, g, or t
nnnnnnnnnnnnnnngcnngactcactagcactctatcacggccatattctggcagggtcagtggctccaactaacatttgtttggtactttacagtttattaaatagatgtttatatggagaagctctcatttctttctcagaagagcctggctaggaaggtggatgaggcaccatattcattttgcaggtgaaattcctgagatgtaaggagctgctgtgacttgctcaaggccttatatcgagtaaacggtagcgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggnnngagatcatgtcctaaccctgatcctcttgtcccacagaattcgggcggagttagggcggagccaatcagcgtgcgccgttccgaaagttgccttttatggctgggcggagaatgggcggtgaacgccgatgantatatnannacgcgccgggtgtggcacagctagttccgtcgcagccgggatttgggtcgcggttcttgtttgtggn

SEQ ID NO: 40 - DNA, 1015 bp, Artificial Sequence
  Synthetic Sequenced PCR product of 3 end of donor DNA plus adjacent TRAC gene exon 1 genomic sequence that included portion of anti-CD38 CAR construct and 650 nt homology arm
  misc_feature (1)..(16): n is a, c, g, or t
  misc_feature (988)..(988): n is a, c, g, or t
nnnnnnnnnnnnnnnnttctgctctacctgggagtggactccggtggtcacgctgagaaaaaagagaaagacccatgaccattccatggtggcggcctcgaggcctgtcaggagaggaaagagaagaaggttagtacaattgtctagatgcattcctagggctgcagggttcatagtgccacttttcctgcactgccccatctcctgcccaccctttcccaggcatagacagtcagtgacttactgtcaagtgacgatcacagggatccacaaacaagaaccgcgacccaaatcccggctgcgacggaactagctgtgccacacccggcgcgtccttatataatcatcggcgttcaccgcccattctccgcccagccataaaaggcaactttcggaacggcgcacgctgattggctccgccctaactccgcccgaattctgtgggacaagaggatcagggttaggacatgatctcatttccctctttgccccaacccaggctggagtccagatgccagtgatggacaagggcggggctctgtggggctggcaagtcacggtctcatgctttatacgggaaatagcatcttagaaaccagctgctcgtgatggactgggactcagggacaggcacaagctatcaatcttggccaagaggccatgatttcagtgaacgttcacggccaggcctggcctgccactcaaggaaacctgaaatgcagggctacttaataatactgcttattcttttatttaataggatcttcttcaaaaccccagcaatataactctggcagagtaaaggcaggcatgggaaaaaggcccagcaaagcaaactgtacatcttggaatctggagtggtctccccaacttaggctgggcattagcagaatgggaggtttatggtatgttggcattaagttgggaaatctatcacattaccaggagattgctctctcattgatagaggttttgaactataaatcanaacacctgcgtctaagccccagcgcta

SEQ ID NO: 41 - DNA, 1035 bp, Artificial Sequence
  Synthetic Sequenced PCR product of 5 end of donor DNA plus adjacent TRAC gene exon 3 genomic sequence that included portion of the anti-CD38 CAR construct and 660 nt homology arm; Exon3 5HA F sequence results
  misc_feature (2)..(21): n is a, c, g, or t
  misc_feature (31)..(31): n is a, c, g, or t
  misc_feature (1030)..(1030): n is a, c, g, or t
  misc_feature (1032)..(1035): n is a, c, g, or t
cnnnnnnnnnnnnnnnnnnnnctttgaggangagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcgaattcgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttgacagtaagtcactgactgtctatgcctgggaaagggtgggcaggagatggggcagtgcaggaaaagtggcactatgaaccctgcagccctaggaatgcatctagacaattgtactaaccntnnnn

SEQ ID NO: 42 - DNA, 1030 bp, Artificial Sequence
  Synthetic Sequenced PCR product of 3 end of donor DNA plus adjacent genomic TRAC gene exon 3 sequence that included portion of the anti-CD38 CAR construct and 650 nt homology arm
  misc_feature (1)..(18): n is a, c, g, or t
  misc_feature (952)..(952): n is a, c, g, or t
  misc_feature (1007)..(1007): n is a, c, g, or t
  misc_feature (1030)..(1030): n is a, c, g, or t
nnnnnnnnnnnnnnnnnngctgcacaggagagtctcagggaccctccaggcttgaccaagcctcccccagactccaccagctgcacctgagagtggacaccggtggtcacggacagaaagaacaggaacacccaggaccactccatggtggcggcctcgaggcctgtcaggagaggaaagagaagaaggttagtacaattgtctagatgcattcctagggctgcagggttcatagtgccacttttcctgcactgccccatctcctgcccaccctttcccaggcatagacagtcagtgacttactgtcaagtgacgatcacagggatccacaaacaagaaccgcgacccaaatcccggctgcgacggaactagctgtgccacacccggcgcgtccttatataatcatcggcgttcaccgcccattctccgcccagccataaaaggcaactttcggaacggcgcacgctgattggctccgccctaactccgcccgaattcgacaggttttgaaagtttaggttcgtatctgtaaaaccaagaggccacagcggttatgggcttgcatgtcccaggcctgcatggcctgcaggacacacccacggttttccccaaggcagagcaggctgggtgggggaggcctgcagctcctgcacctcctgtcccttgcagcttctgtgcatatccccttacaaacagattctgtgcattcttccagggccaacagtattgtgtttcaagacaggaaacaccgactagacttggctcttattttatttctgagcccttctggggatggggtaggtgctcagaatttatcatcacaatacctcgggtccagatgggccccttagtgagttccgctaccacaggaaggatgactcaataagaacaactcaaatggaaatgaatatgggcccagggtgggtgtgaggagtggcaggggattcttganagacaggaccattgcccacagcctatgtgagtactgttgcttgtcttgaaagaangcaaaacctcttggctgtcctcn

SEQ ID NO: 43 - DNA, 1015 bp, Artificial Sequence
  Synthetic Sequenced PCR product of 3 end of donor DNA plus adjacent PD-1 genomic sequence that included portion of the anti-CD38 CAR construct and 660 nt homology arm
  misc_feature (1)..(19): n is a, c, g, or t
  misc_feature (22)..(22): n is a, c, g, or t
  misc_feature (32)..(32): n is a, c, g, or t
  misc_feature (925)..(926): n is a, c, g, or t
  misc_feature (930)..(930): n is a, c, g, or t
  misc_feature (935)..(936): n is a, c, g, or t
  misc_feature (943)..(943): n is a, c, g, or t
  misc_feature (947)..(947): n is a, c, g, or t
  misc_feature (960)..(961): n is a, c, g, or t
  misc_feature (971)..(971): n is a, c, g, or t
  misc_feature (974)..(976): n is a, c, g, or t
  misc_feature (978)..(978): n is a, c, g, or t
  misc_feature (983)..(985): n is a, c, g, or t
  misc_feature (990)..(991): n is a, c, g, or t
  misc_feature (999)..(999): n is a, c, g, or t
  misc_feature (1012)..(1013): n is a, c, g, or t
  misc_feature (1015)..(1015): n is a, c, g, or t
nnnnnnnnnnnnnnnnnnntcntgctggtganggtggcctttattattttctgggttcgatctaagcgaagccgcttgttgcattctgactacatgaatatgacgccaagacggccagggccaacaagaaagcattaccaaccgtacgcccccccgcgagactacgcggcctaccgcagcagggtaaaatttagcaggtctgcagatgcgcctgcgtatcaacagggtcagaatcagctctataatgagctgaacctcgggcggcgggaagagtatgatgttctcgataaaaggagaggacgagaccccgaaatgggcggcaaaccgagacgcaaaaatcctcaggaggggctctacaatgaacttcaaaaagacaaaatggccgaagcatactcagaaatcggaatgaaaggggagaggagacgcgggaagggccatgatggactgtatcagggactttccacagccaccaaggacacctatgacgctctccacatgcaggcgctgccgcctagatgataaaattgttgttgttaacttgtttattgcagcttataatggttacaaataaagcaatagcatcacaaatttcacaaataaagcatttttttcactgcattctagttgtggtttgtccaaactcatcaatgtatcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcaaactcctgggttcctctctggnncccnactcnncccctgncccnagtcccctctttnntcctgggcangcnnnanctctnnnccctnncagccggnccttggggctgcnngn

(19) United States
(12) Patent Application Publication
(10) Pub. No.: US 2020/0224160 A1
(43) Pub. Date: Jul. 16, 2020

(54) PROCESS FOR DNA INTEGRATION USING RNA-GUIDED ENDONUCLEASES

(71) Applicant: Sorrento Therapeutics, Inc., San Diego, CA (US)

(72) Inventors: Beibei Ding, San Diego, CA (US); Wenzhong Guo, San Diego, CA (US); Yanliang Zhang, San Diego, CA (US)

(21) Appl. No.: 16/288,052

(22) Filed: Feb. 27, 2019

Related U.S. Application Data
(60) Provisional application No. 62/635,702, filed on Feb. 27, 2018.

(51) Int. Cl. C12N 5/0783; C12N 9/22; C12N 15/90; C12N 15/62; C07K 14/725

(52) U.S. Cl. CPC C12N 5/0636; C12N 9/22; C12N 15/907; C12N 2800/30; C07K 14/7051; C07K 2319/03; C12N 2800/70; C12N 15/62

(57) ABSTRACT

There is disclosed an improved, safer and commercially efficient process for developing genetically engineered cells. More specifically, there is disclosed a process comprises introducing a donor DNA construct, a guide RNA, and an RNA-guided nuclease with the host cells to be transfected; and introducing the three components into the host cell. There is further disclosed a donor DNA construct designed for inserting a CAR (chimeric antigen receptor) into a defined genomic site of a host cell. Further, the present disclosure provides a host cell transfected with a CAR that lacks viral vectors that can present a safety concern. The disclosure provides for more efficient and more cost-effective process for engineering T cells to express CAR constructs.

This application claims priority to U.S. Provisional Patent Application No. 62/635,702 filed Feb. 27, 2018, which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Nov. 12, 2019, is named 087735_0103_ST25.TXT and is 52,000 bytes in size.

### TECHNICAL FIELD

The present disclosure provides methods and compositions for efficiently integrating a DNA sequence of interest into a target DNA molecule, such as a host genome using an RNA-guided endonuclease such as a cas protein.

### BACKGROUND

Targeted integration of an exogenous DNA sequence into a genomic locus has been highly desired. CRISPR-Cas genome engineering is a fast and relatively simple way to knockout gene function, or precisely knock-in a DNA sequence for gene correction or gene tagging. Targeted gene knockout is achieved through generation of a double-strand break (DSB) in the DNA using Cas9 nuclease and guide RNA (gRNA). The DSB is then repaired, often imperfectly, by random insertions or deletions (indels), through the endogenous non-homologous end joining (NHEJ) repair pathway. For knock-in experiments, in addition to the Cas9 nuclease and gRNA, a DNA donor template is required and the DSB is repaired with the donor template typically through the homology-directed repair (HDR) pathway.

Knock-in using a donor template, either a single-stranded DNA (ssDNA) donor oligo or donor plasmid (dsDNA), has a relatively low efficiency, often in the 1-10% range. Therefore, successful HDR-mediated knock-in experiments require important design considerations and experimental optimization. Using single-stranded oligodeoxynucleotides (ssODNs) with short homology arms, several groups have achieved precise DNA editing such as SNP correction or epitope tag addition. A donor plasmid (dsDNA) is able to integrate much longer exogenous DNA, however efficiency is very low. Several groups used an AAV (viral) vector to provide HDR donor ssDNA and combined with CRISPR/Cas9 to achieve 40-60% gene knock-in efficiency. However, these methods still need to produce high titer AAV vectors which is time-consuming and needs to be compatible with cGMP production for clinical application.

A genome engineering tool has been developed based on the components of the type II prokaryotic CRISPR (Clustered Regularly Interspaced Short palindromic Repeats) adaptive immune system of some bacteria such as S. pyogenes. This multi-component system referred to as RNA-guided Cas nuclease system or more simply as CRISPR, involves a Cas endonuclease, coupled with a guide RNA molecule, that have the ability to create double-stranded breaks in genomic DNA at specific sequences that are targeted by the guide RNA. The RNA-guided Cas endonuclease has the ability to cleave the DNA where the RNA guide hybridizes to the genome sequence. Additionally, the Cas9 nuclease cuts the DNA only if a specific sequence known as protospacer adjacent motif (PAM) is present immediately downstream of the target sequence in the genome. The canonical PAM sequence in S. pyogenes is 5′-NGG-3′, where N refers to any nucleotide.

It has been demonstrated that the expression of a single chimeric crRNA:tracrRNA transcript, which normally is expressed as two different RNAs in the native type II CRISPR system, is sufficient to direct the Cas9 nuclease to sequence-specifically cleave target DNA sequences. In addition, several mutant forms of Cas9 nuclease have been developed. For instance, one mutant form of Cas9 nuclease functions as a nickase, generating a break in complementary strand of DNA rather than both strands as with the wild-type Cas9. This allows repair of the DNA template using a high-fidelity pathway rather than NHEJ, which prevents formation of indels at the targeted locus, and possibly other locations in the genome to reduce possible off-target/toxicity effects while maintaining ability to undergo homologous recombination. Paired nicking can reduce off-target activity by 50- to 1,500-fold in cell lines and to facilitate gene knockout in mouse zygote without losing on-target cleavage efficiency.

In addition, cas proteins have been isolated from a variety of bacteria and have been found to use different PAM sequences than S. pyogenes cas9. In addition, some cas proteins such as cas12a naturally use a single RNA guide that is, they use a crRNA that hybridizes to a target sequence but do not use a tracrRNA.

Adoptive immunotherapy involves transfer of autologous antigen-specific cells generated ex vivo, is a promising strategy to treat viral infections and cancer. The cells used for adoptive immunotherapy can be generated either by expansion of antigen-specific cells or redirection of cells through genetic engineering.

CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), and 4-1BB (CD137) have been added alone (second generation) or in combination (third generation) to enhance survival and increase proliferation of CAR modified cell. CARs have successfully allowed T cells to be redirected against antigens expressed at the surface of tumor cells from various malignancies including lymphomas and solid tumors,

CAR (chimeric antigen receptor) cell immunotherapy, which involves removing T-cells from a patient's blood, adding a CAR through gene transfer, and infusing the genetically engineered cells back into the body, is one of the most promising methods in treating cancer. Currently, the gene transfer techniques include viral-based gene transfer methods using gamma-retroviral vectors or lentiviral vectors. To make GMP (FDA's required good manufacturing practice regulations) level viral-vector, the viral vector has to comply with clinical safety standards such as replication incompetence, low genotoxicity, and low immunogenicity. These conventional approaches have ease of use and reasonable expression, however they can give rise to secondary transformation events, e.g., unwanted blood cancers and other events resulting from viral genome integration into the T cells.

A review article (Ren and Zhao, Protein Cell 8(9):634-643, 2017) indicates that any use of CRISPR/Cas9 still involves the use of viral vector for a knocking in process to insert a CAR (chimeric antigen receptor) construct into a T cell genome. “Gene editing with CRISPR encoded by non-integrating virus, such as adenovirus and adenovirus-associated virus (AAV), has also been reported.” In addition, Ren et al., Clin. Cancer Res. 16:1300, published online 4 Nov. 2016 used a CD19 CAR construct and found that gene disruption in T cells is not very efficient with lentiviral and adenoviral CRISPR.

Although RNA-guided endonucleases, such as the Cas9/CRISPR system, appear to be an attractive approach for genetically engineering some mammalian cells, the use of Cas9/CRISPR in primary cells, in particular in T cells, is significantly more difficult because: (1) T-cells are adversely affected by the introduction of DNA in their cytoplasm: high rate of apoptosis is observed when transforming cells with DNA vectors; (2) the CRISPR system requires stable expression of Cas9 in the cells, however, prolonged expression of Cas9 in living cells may lead to chromosomal defects; and (3) the specificity of current RNA-guided endonuclease is determined only by sequences comprising 11 nucleotides (N12-20NGG, where NGG represents the PAM), which makes it very difficult to identify target sequences in desired loci that are unique in the genome. Other nucleases, in addition to CASA, are zinc linger nucleases (ZFN) or TAL effector nucleases (TALEN)

The present disclosure aims to provide solutions to these limitations in order to efficiently implement RNA-guided endonuclease engineering in host cells such as T cells. There is a need in the art for safer transduction techniques for Chimeric Antigen Receptor constructs that do not include transduction with viral vectors but instead can use transfection techniques. This includes increasing CAR construct transfection efficiency, while avoiding the risk of having viral genes potentially expressed by the transduced cells that are administered to a patient. The present disclosure was made to address this need in the art.

### SUMMARY

The present disclosure provides an improved, safer, and commercially efficient process for developing genetically engineered and transduced cells for immunotherapy. More specifically, the disclosed process comprises introducing an RNA-guided endonuclease, a guide RNA, and a donor DNA construct into host cells, where the guide RNA is engineered to direct the cas protein with which it is complexed to a targeted site of the host genome. Cleavage of the genomic DNA at the target site by the RNA-guided endonuclease and subsequent repair of the double stranded break using the donor fragment that includes homology arms by homology- directed repair (HDR) results in integration of sequences of the donor DNA molecule positioned between the homology arms. The method can be used to simultaneously knock out a gene at the target locus and insert or “knock in” at the disrupted locus a transgene that is provided in the donor DNA molecule. The method can be used on any host cells, including prokaryotic and eukaryotic cells, and can be used with mammalian cells, such as human cells. The method has advantages in ease of use, efficiency, and the ability to generate genome modifications that do not entail the use of selectable markers or viral vectors that are undesirable in many applications, including clinical applications. In some embodiments, the host cells are hematopoietic cells, such as, for example, T cells.

The present disclosure also provides donor DNA compositions, where the donor DNA molecule includes one or more modifications to nucleotides of one donor DNA strand. The donor DNA can include homology arms flanking a sequence of interest whose integration into the host genome is desired, where the homology arms have sequences homologous to sequences occurring in the host genome on either side of the target sequence. The donor DNA in some embodiments is double-stranded. In various embodiments the donor DNA includes from one to ten modified nucleotides that are proximal to the 5′ end of one strand of the donor DNA, for example, that occur within ten nucleotides or within five nucleotides of the 5′ terminus of one strand of the donor DNA. In some embodiments the donor DNA has at least two types of nucleic acid modification of from one to ten nucleotides at the 5′ end of one strand of the donor DNA. In some embodiments the donor DNA has two types of nucleic acid modification of from one to ten nucleotides at the 5′ end of one strand of the donor DNA. The modification may be, for example, phosphorothioate (PS) linkages between nucleotides, or may be 2′-O-methylation of the deoxyribose of one or more nucleotides of the donor DNA molecule. For example, a donor DNA molecule can have one, two, three or four PS bonds within the first five, first six, or first seven nucleotides from the 5′ end of the modified strand and can also have one, two, three or four 2′-O-methyl modified nucleotides within the first five, first six, or first seven nucleotides from the 5′ end of the modified strand. In some embodiments the donor DNA molecule is double-stranded and one strand comprises the modifications at the 5′ end. In some embodiments the donor DNA molecule is double-stranded and one strand has two or more modifications on any of the first ten or first five nucleotides from the 5′ end and the opposite strand has a terminal 5′ phosphate. In various embodiments, the donor DNA molecule is double-stranded and has at least two PS bonds and at least two 2′O-methyl-modified nucleotides on one strand of the donor DNA, where the PS and 2′-O methyl modifications occur within the first five nucleotides from the 5′ end of the modified strand. In various embodiments, the donor DNA molecule is double-stranded and has three PS bonds and three 2′O-methyl-modified nucleotides on one strand of the donor DNA, where the PS and 2′-O methyl modifications occur within the first five nucleotides from the 5′ end of the modified strand. In some examples of these embodiments, the opposite strand includes a terminal 5′ phosphate. The donor DNA is introduced into the cell as a double-stranded molecule.

The present disclosure further provides a donor DNA construct designed for inserting a CAR (chimeric antigen receptor) into a host cell. Further, the present disclosure provides a host cell transduced with a CAR that lacks viral vectors. The disclosure provides for more efficient and more cost-effective process for engineering T cells to express CAR constructs. The CAR construct can include homology arms that target the construct to a T cell receptor gene, PD-1 gene, or TIM3 gene, as nonlimiting examples, for simultaneous knock-in of the CAR construct and knock out of the TCR, PD-1, or TIM3 gene.

In a further aspect, provided herein is a system for genome modification that comprises: an RNA-guide endonuclease or a nucleic acid molecule encoding an RNA-guide endonuclease; a guide RNA or a nucleic acid molecule encoding a guide RNA; and a donor DNA molecule, where the donor DNA molecule includes at least one nucleotide modification within ten or within five nucleotides of the 5′ terminus. In some embodiments the donor DNA is double-stranded and includes at least one, at least two, or at least three modifications on at least one, at least two, or at least three nucleotides occurring within ten or within five nucleotides of one strand of the double stranded donor molecule. The modifications can be, for example, phosphorothioate bonds and/or 2′-O methylation of nucleotides. The donor DNA can have homology arms flanking a sequence of interest to be integrated into the genome. The sequence of interest can be an expression cassette, for example, for expression a construct that includes one or more antibody or receptor domains. Homology arms can be between about 50 and about 5000 nucleotides in length, or between about 100 and 1000 nucleotides in length, for example between about 150 and about 800 nucleotides in length.

In some embodiments, the nuclease is selected from the group consisting of Cas9, Cas12a, Cas12b, CasX, and combinations thereof. The guide RNA can be a chimeric guide, having sequences of both crRNA and tracrRNA, or can be a crRNA, and can optionally include one or more phosphorothioate (PS) oligonucleotides. Where the guide is a crRNA, and the RNA-guided endonuclease uses a tracrRNA, the system can also include a tracrRNA. For example, Cas9 can be used with a crRNA and a tracrRNA or can be used with a chimeric guide RNA (sometimes called a single guide or “sgRNA”) that combines structural features of the crRNA and tracrRNA. Cas12a on the other hand naturally uses only a crRNA and has no associated tracrRNA. In various embodiments, the RNA-guide endonuclease, guide RNA (that can be a crRNA or a chimeric guide RNA), and, when included, tracr RNA, can be complexed as a ribonucleoprotein complex that is introduced to the cell. The donor DNA can be introduced into the target cell together with the RNP, or separately, for example, in a separate electroporation or transfection.

Also provided herein is a method for site-specific integration of a donor DNA into a target DNA molecule, where the method includes introducing into a cell: an RNA-guided endonuclease or a nucleic acid molecule encoding an RNA-guided endonuclease; at least one engineered guide RNA or at least one nucleic acid molecule encoding an engineered guide RNA; and a donor DNA molecule comprising at least one nucleic acid modification; where the guide RNA comprises a target sequence designed to hybridize with a target site in the target DNA and the donor DNA is inserted into the target DNA molecule at the target site. In various embodiments the donor DNA includes at least two modified nucleases, which can have the same or different modifications, and preferably occur within ten or within five nucleotides of the 5′ terminus of one strand of the donor DNA. In some embodiments, the donor DNA is double-stranded and the one or more nucleotide modifications occur on a single strand of the donor DNA molecule. In some embodiments, the donor DNA is double-stranded and the one or more nucleotide modifications occur on a single strand of the donor DNA molecule within ten or within five nucleotides of the 5′ terminus of the modified strand. In some embodiments, the donor DNA includes a backbone modification such as a phosphoramidite or phoshorothioate modification. In some embodiments, the donor DNA includes a modification of a sugar moiety of a nucleotide. In some embodiments, the donor DNA is double stranded and includes at least one, at least two, or at least three phosphorothioate modifications within five nucleotides of the 5′ end of a single strand of the donor DNA molecule and further includes at least one, at least two, or at least three 2′-O-methylated nucleotides within five nucleotides of the 5′ end of a single strand of the donor DNA molecule. In various embodiments the donor DNA includes homology arms flanking a DNA sequence of interest, such as, for example, an expression cassette, where the homology arms have homology to sites in the target genome on either side of the target site of the RNA-guide endonuclease. Homology arms can be from about 50 to about 2000 nt in length, and may be, for example between 100 and 1000 nt in length, or between 150 and 650 nt in length, for example, between 150 and 350 nt in length, or 150 to 200 nt in length. In various embodiments a donor DNA molecule has two or more nucleotide modifications on the modified strand and the opposite strand includes a terminal phosphate.

The RNA-guided endonuclease can be a cas protein and can be, as nonlimiting example, a cas9, cas12a, or casX protein. In various embodiments of the method, the RNA-guided endonuclease and an RNA guide are introduced into the cell as a ribonucleoprotein complex (RNP). The RNP can in some embodiments further include a tracr RNA. An RNP can be introduced into a target cell by any feasible means, including electroporation or liposome transfer, for example. The donor DNA can be delivered to the cell simultaneously with the RNP, or separately.

Also included herein are methods of producing a donor DNA molecule, where the method includes amplifying a template DNA that includes homology arms flanking a sequence of interest using a first primer that includes at least two nucleotide modifications within the first five nucleotides of the 5′ terminus of the primer, and a second primer that includes a 5′ terminal phosphate. In various embodiments the first primer can include at least two, at least three, at least four, at least five, at least six, at least seven, at least eight, at least nine, or at least ten modifications, and can include more than one type of modification. For example, a primer for producing a donor DNA molecule can include at least one phosphorothioate modification and at least one 2′O-methyl modification of a nucleotide within five nucleotides of the 5′ terminus of the primer.

### BRIEF DESCRIPTION OF THE FIGURES

FIG. 1A provides chemical drawings that show, in the right structure, a phosphorothioate (PS) modification of the bond between nucleotides as they might occur in a primer. The nucleotides shown in the oligonucleotide on the left are attached via a (nonmodified) phosphodiester bond. FIG. 1B provides a chemical drawing of an oligonucleotide having two PS bonds that join the 5′-most nucleotide to the next nucleotide “downstream” in the oligonucleotide, which in turn is attached to the following downstream nucleotide of the oligonucleotide by a PS bond. The 5′-most nucleotide of the oligonucleotide includes a 2′ O-methyl modification.

FIG. 2A is a diagram of a CAR donor DNA construct that includes an open reading frame having a sequence encoding a single chain variable fragment (scFv), followed by the CD8a leader peptide which is then followed by a CD28 hinge-CD28 transmembrane-intracellular regions and then a CD3 zeta intracellular domain. The coding sequence is preceded by a JeT promoter (SEQ ID NO:3) and the construct includes homology arms (HA), in this case matching sequences of the human TRAC locus, flanking the promoter plus coding sequences. shows the structure of the donor DNA construct (top) and primer design for confirming right knock in (bottom). This provides a diagram of the template DNA used for generating donor DNA. The anti-CD38A2 contains a CD38 CAR transgene with expression driven by the JeT promoter and flanked by homology arms on the 5′ and 3′ sides to enable targeted integration. FIG. 2B shows the same diagram indicating the positions of PCR primers used to confirm CAR integration by amplification with one primer located within the CAR and one primer in TRAC outside of the homology arms at both the 5′ and 3′ ends to generate 1371-bp and 1591-bp products, respectively, when integration is at the targeted integration site.

FIG. 3A provides flow cytometry plots of PBMCs 8 days after transformation with a donor DNA that included a construct for expressing an anti-CD38 CAR and an RNP comprising a guide RNA targeting the TRAC locus. The CAR cassette was flanked by homology arms having homology to TRAC locus sequences flanking the integration target site in exon 1 of the TRAC gene. The Y axis reports cell size. Anti-CD38 construct expression is along the x axis.

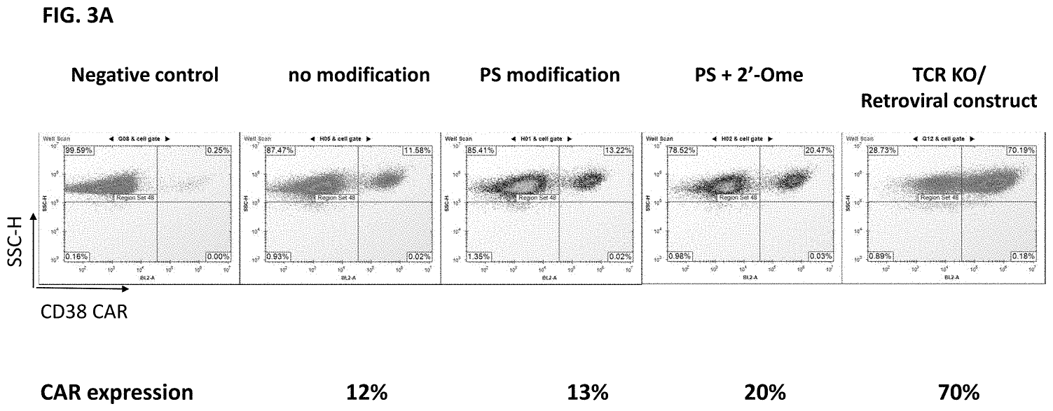

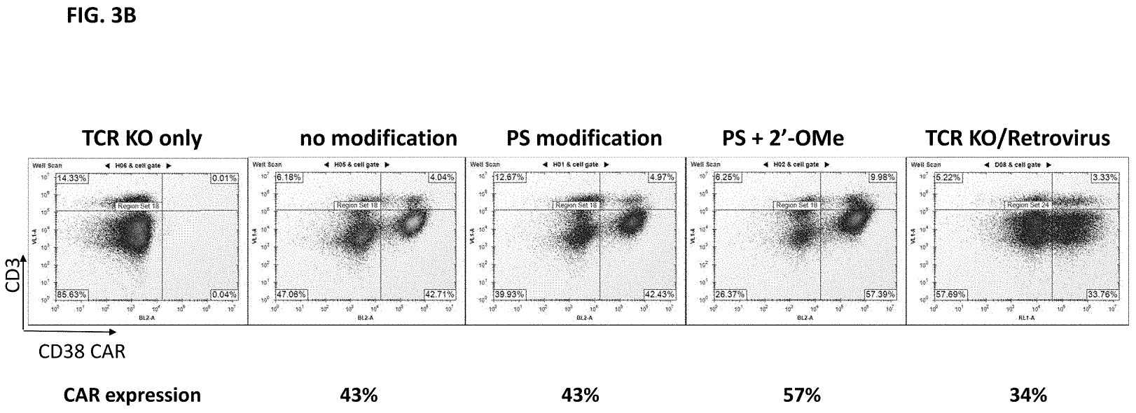

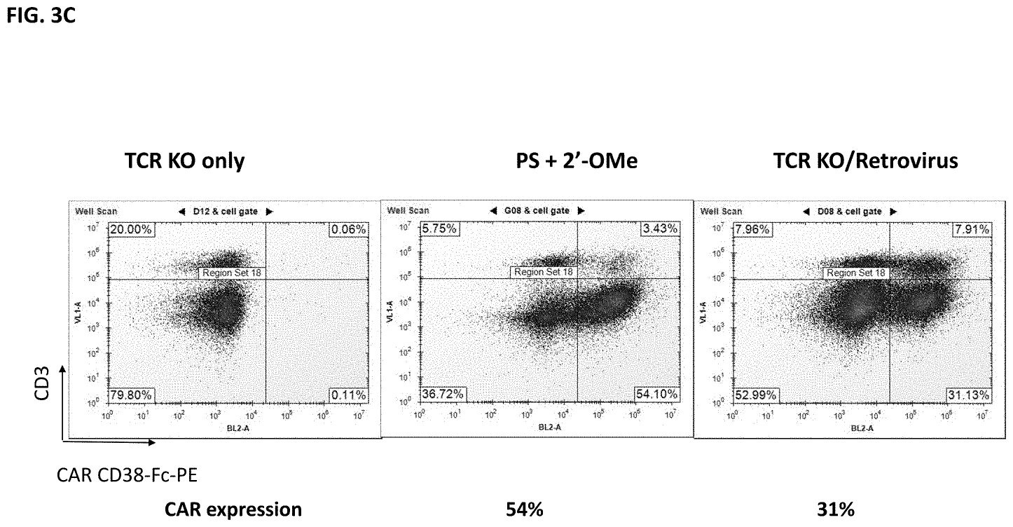

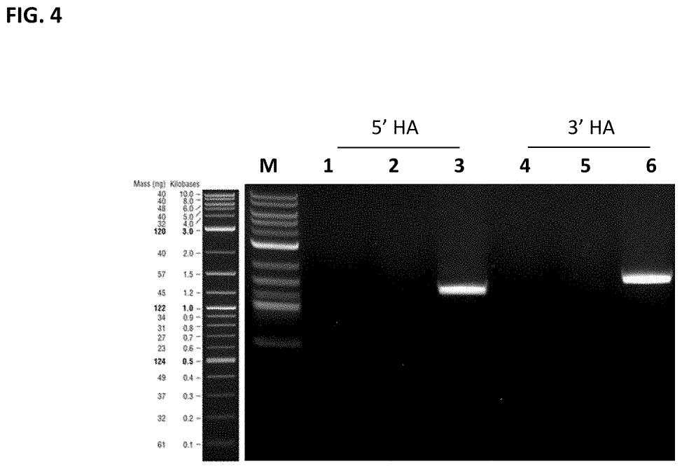

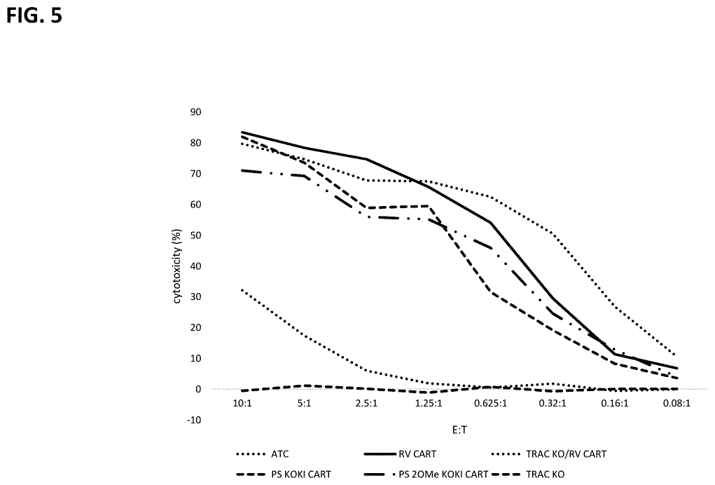

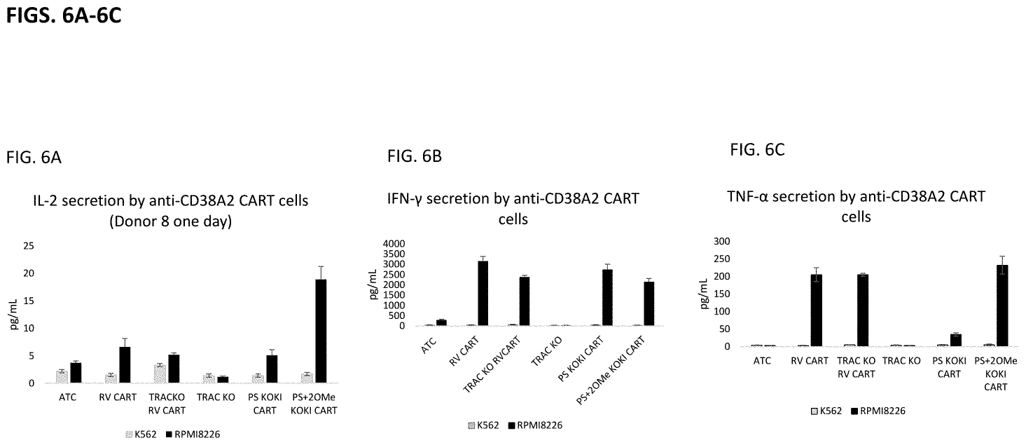

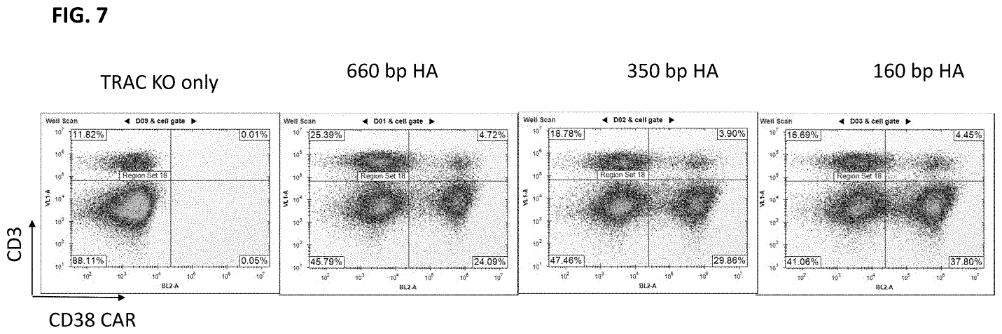

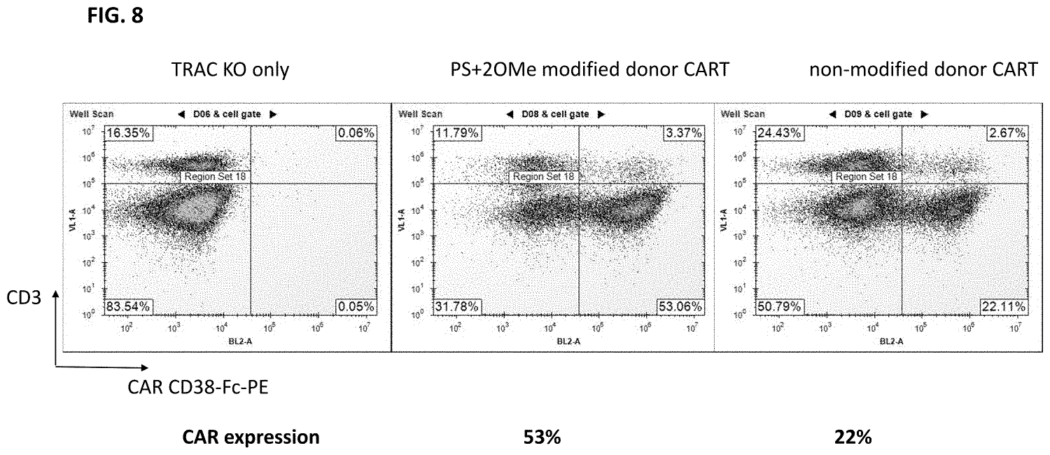

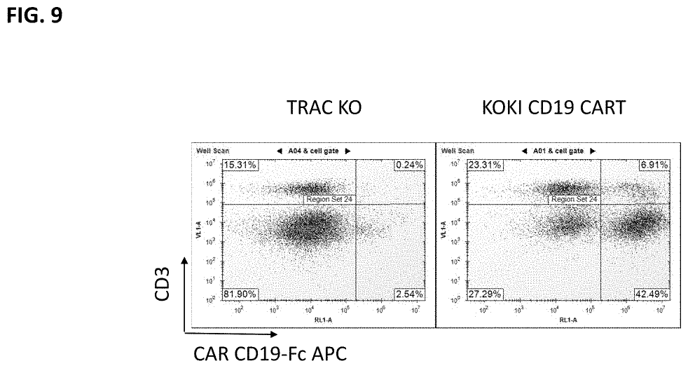

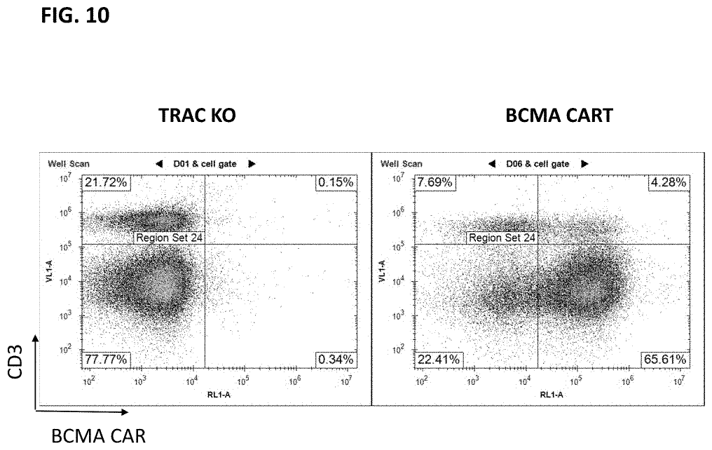

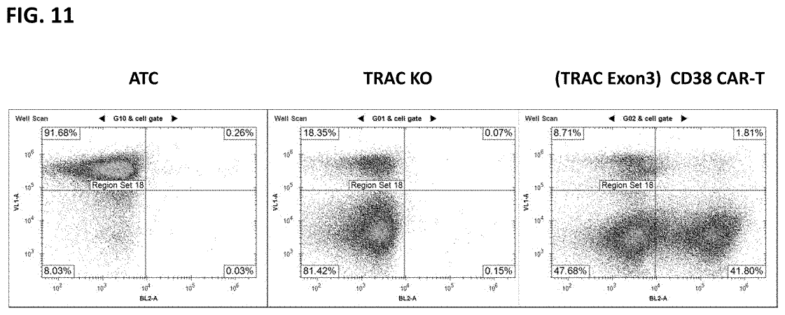

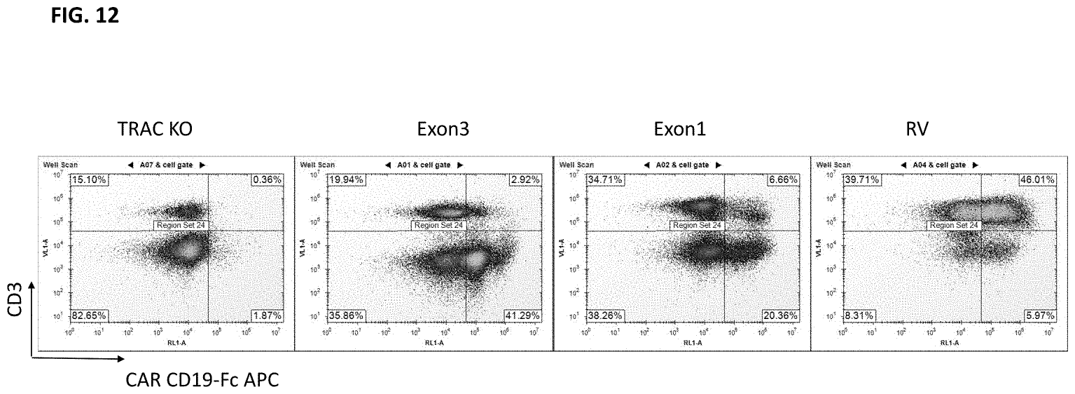

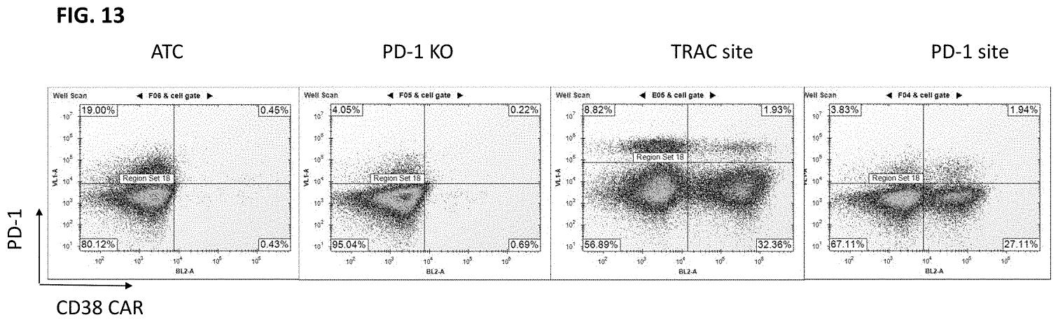

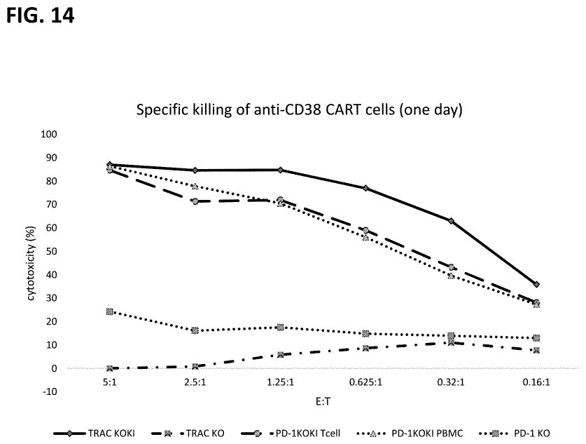

Negative control: no donor DNA was transformed into the target cells; No modification—the donor DNA had no chemical modifications; PS modification: three phosphorothioate bonds occurred within the 5′-most five nucleotide backbone positions; PS +2′-OMe: in addition to phosphorothioate bonds, the three nucleotides within the 5′-most five nucleotides of the donor included 2′-OMe in addition to PS modifications; TCR KO/retroviral construct: the cells were transfected with the RNP in the absence of donor DNA to knock out the TCR gene and transduced with a retrovirus to express the anti-CD38 CAR. FIG. 3B provides the results of flow cytometry performed on the same cultures as in A) ten days after transfection. FIG. 3C provides the results of flow cytometry performed on the culture that received the doubly-modified donor DNA and control (TRAC knockout only and TRAC knockout with retroviral transduction) twenty days after transfection.

FIG. 4 shows a gel of PCR products showing integration of the donor DNA at the targeted TRAC (Exon1) site. Primary human T cells were electroporated with TRAC RNP only or together with ssDNA. PCR was used to confirm the presence of the anti-CD38A2 CAR transgene integrated in the TRAC locus two weeks post-electroporation (lanes 3 and 6, depicting products from 5′ and 3′ integration regions). No bands were observed in non-transformed ATCs (lanes 1 and 4) or T cells that were transformed with the TRAC exon 1 targeting RNP but did not receive the donor DNA (lanes 2 and 5).

FIG. 5 is a graph showing cytotoxicity assay results with Activated T cells (ATCs, stars) as a control, TCR knock out ATC, anti-CD38A2 retrovirus transduced CART cells RV CART, black line), TRAC knock out retrovirus transduced CART cells (dots), TRAC knock out together with phosphorothioate modified ss donor DNA knock in (dashes), TRAC knock out together with phosphorothioate and 2′ O-Methyl modified ssDNA knock in (dashes and dots).

FIGS. 6A-6C provide graphs of the results of cytokine secretion assays using anti-CD38 CART cells and controls co-cultured with K52 or RPM18226 cells. The T cell cultures tested are as provided in FIG. 5.

FIG. 7 provides the results of testing donor DNAs having homology arms (HAs) of different lengths. Cultures were assessed by flow cytometry for loss of TCR expression (Y axis) and anti-CD38 expression (X axis).

FIG. 8 provides the results of testing double stranded donor DNAs modified by the addition of three PS bonds and three 2′O methyl nucleotides proximal to the 5′ end of one strand of the donor DNA molecule. Cultures were assessed by flow cytometry for loss of TCR expression (Y axis) and anti-CD38 expression (X axis).

FIG. 9 provides the results of flow cytometry on cells transfected with a ds PS and 2′-OMe-modified donor DNA that included a cassette for expressing an anti-CD19 CAR. The donor was directed to the TRAC exon 1 locus by cotransfection with an RNP. TCR expression is determined on the Y axis and anti-CD19 CAR expression on the Y axis.

FIG. 10 provides the results of flow cytometry on cells transfected with a ds PS and 2′-OMe-modified donor DNA that included a cassette for expressing an anti-BCMA CAR. The donor was directed to the TRAC exon 1 locus by cotransfection with an RNP. TCR expression is determined on the Y axis and anti-BCMA CAR expression on the Y axis.

FIG. 11 provides the results of flow cytometry on cells transfected with a ds PS and 2′-OMe-modified donor DNA that included a cassette for expressing an anti-CD38 CAR. The donor was directed to the TRAC exon 3 locus by cotransfection with an RNP. TCR expression is determined on the Y axis and anti-CD38 CAR expression on the Y axis.

FIG. 12 provides the results of flow cytometry on cells transfected with a ds PS and 2′-OMe-modified donor DNA that included a cassette for expressing an anti-CD19 CAR. In one culture, the donor had homology arms derived from TRAC exon 3 was directed to the TRAC exon 3 locus by cotransfection with an RNP having an exon 3 guide RNA (2^{nd} panel). In another culture, the donor had homology arms derived from TRAC exon 1 was directed to the TRAC exon 1 locus by cotransfection with an RNP having an exon 1 guide RNA (2^{nd} panel). TCR expression is determined on the Y axis and anti-CD19 CAR expression on the Y axis.

FIG. 13 provides the results of flow cytometry on cells transfected with a ds PS and 2′-OMe-modified donor DNA that included a cassette for expressing an anti-C38 CAR and homology arms derived from the TRAC gene or the PD-1 gene. In one culture, the donor had homology arms derived from TRAC exon 1 was directed to the TRAC exon 1 locus by cotransfection with an RNP having an exon 1 guide RNA (3rd panel). In another culture, the donor had homology arms derived from the PD-1 locus and was directed to the PD-1 gene by cotransfection with an RNP having a PD-I gene guide RNA (4th panel). TCR expression is determined on the Y axis and anti-CD38 or anti-PD-1 CAR expression on the Y axis.

FIG. 14 provides the results of cytotoxicity assays using T cell cultures that were transfected with doubly modified (PS and 2′-OMe) donor fragment that included and anti-CD38 CAR construct and PD-1 gene-derived homology arms was targeted to the PD-1 gene by an RNP that included a guide RNA having a target sequence from the PD-1 gene.

### DETAILED DESCRIPTION

### Definitions

Unless specifically indicated otherwise, all technical and scientific terms used herein have the same meaning as commonly understood by those of ordinary skill in the art. In addition, any method or material similar or equivalent to a method or material described herein can be used in the practice of the present disclosure.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “primary cell” refers to a cell isolated directly from a multicellular organism. Primary cells typically have undergone very few population doublings and are therefore more representative of the main functional component of the tissue from which they are derived in comparison to continuous (tumor or artificially immortalized) cell lines. In some cases, primary cells are cells that have been isolated and then used immediately. In other cases, primary cells cannot divide indefinitely and thus cannot be cultured for long periods of time in vitro.

The term “genome editing” refers to a type of genetic engineering in which DNA is inserted, replaced, or removed from a target DNA, e.g., the genome of a cell, using one or more nucleases. The nucleases create specific double-strand breaks (DSBs) at desired locations in a genome and harness a cell's endogenous mechanisms to repair the induced break by homology-directed repair (HDR) (e.g., homologous recombination) or by nonhomologous end joining (NHEJ). Any suitable nuclease can be introduced into a cell to induce genome editing of a target DNA sequence including, but not limited to, CRISPR-associated protein (Cas) nucleases, zinc finger nucleases (ZENs), transcription activator-like effector nucleases (TALENs), meganucleases, other endo- or exo-nucleases, variants thereof, fragments thereof, and combinations thereof. Nuclease-mediated genome editing of a target DNA sequence can be “induced” or “modulated” (e.g., enhanced) using the modified single guide RNAs (sgRNAs) described herein in combination with Cas nucleases (e.g., Cas9 polypeptides or Cas9 mRNA), to improve the efficiency of precise genome editing via homology-directed repair (HDR).

The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA.

The term “nonhomologous end joining” or “MID” refers to a pathway that repairs double-strand DNA breaks in which the break ends are directly ligated without the need for a homologous template.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. The term also encompasses nucleic acids containing known analogs of natural nucleotides that have similar binding properties as the reference nucleic acid and are metabolized in a manner similar to naturally occurring nucleotides. A particular nucleic acid sequence also encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al, Nucleic Acid Res. 19:5081 (1991); Ohtsuka et al, J. Biol. Chem. 260:2605-2608 (1985); and Rossolini et al, Mol. Cell. Probes 8:91-98 (1994)). The term nucleic acid is used interchangeably with gene, cDNA, and mRNA encoded by a gene.

The term “nucleotide analog” or “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G), in or on the sugar moiety of the nucleoside (e.g., ribose, deoxyribose, modified ribose, modified deoxyribose, six-membered sugar analog, or open-chain sugar analog), or the phosphate.

The term “gene” or “nucleotide sequence encoding a polypeptide” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons),

The terms “polypeptide,” “peptide,” and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms encompass amino acid chains of any length, including full-length proteins, wherein the amino acid residues are linked by covalent peptide bonds.

The term “variant” refers to a form of an organism, strain, gene, polynucleotide, polypeptide, or characteristic that deviates from what occurs in nature.

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15. 16, 17, 18, 19, 20, 21, 22, 23. 24, 25, 30, 35, 40, 45, 50, or more nucleotides, or refers to two nucleic acids that hybridize under stringent conditions.

The term “stringent conditions” for hybridization refers to conditions under which a nucleic acid having complementarily to a target sequence predominantly hybridizes with the target sequence, and substantially does not hybridize to non-target sequences. Stringent conditions are generally sequence-dependent and vary depending on a number of factors. in general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology—Hybridization With Nucleic Acid Probes Part 1, Second Chapter “Overview of principles of hybridization and the strategy of nucleic acid probe assay”, Elsevier, N.Y.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these.

A “recombinant expression vector” is a nucleic acid construct, generated recombinantly OF synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter.

“Operably linked” means two or more genetic elements, such as a polynucleotide coding sequence and a promoter, placed in relative positions that permit the proper biological functioning of the elements, such as the promoter directing transcription of the coding sequence.

The term “promoter” refers to an array of nucleic acid control sequences that direct transcription of a nucleic acid. As used herein, a promoter includes necessary nucleic acid sequences near the start site of transcription, such as, in the case of a polymerase 11 type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators), as well as those that confer certain binding affinity or antigenicity to the recombinant protein produced from the expression vector. “Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al, Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide).

The term “single nucleotide polymorphism” or “SNP” refers to a change of a single nucleotide with a polynucleotide, including within an allele. This can include the replacement of one nucleotide by another, as well as deletion or insertion of a single nucleotide. Most typically, SNPs are biallelic markers although tri- and tetra-allelic markers can also exist. By way of non-limiting example, a nucleic acid molecule comprising SNP AT may include a C or A at the polymorphic position.

The terms “culture,” “culturing,” “grow,” “growing,” “maintain,” “maintaining,” “expand,” “expanding,” etc., when referring to cell culture itself or the process of culturing, can be used interchangeably to mean that a cell (e.g., primary cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

The term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent (e.g., Cas nuclease, modified single guide RNA, etc.) that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other agents, timing of administration, and the physical delivery system in which it is carried.

The term “pharmaceutically acceptable carrier” refers to a substance that aids the administration of an agent (e.g., Cas nuclease, modified single guide RNA, etc.) to a cell, an organism, or a subject. “Pharmaceutically acceptable carrier” refers to a carrier or excipient that can be included in a composition or formulation and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical carriers are useful in the present invention.

The term “increasing stability,” with respect to components of the CRISPR system, refers to modifications that stabilize the structure of any molecular component of the CRISPR system. The term includes modifications that decrease, inhibit, diminish, or reduce the degradation of any molecular component of the CRISPR system.

The term “increasing specificity,” with respect to components of the CRISPR system, refers to modifications that increase the specific activity (e.g., the on-target activity) of any molecular component of the CRISPR system. The term includes modifications that decrease, inhibit, diminish, or reduce the non-specific activity (e.g., the off-target activity) of any molecular component of the CRISPR system.

The term “decreasing toxicity,” with respect to components of the CRISPR system, refers to modifications that decrease, inhibit, diminish, or reduce the toxic effect of any molecular component of the CRISPR system on a cell, organism, subject, and the like.

The term “enhanced activity,” with respect to components of the CRISPR system and in the context of gene regulation, refers to an increase or improvement in the efficiency and/or the frequency of inducing, modulating, regulating, or controlling genome editing and/or gene expression.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value IV.

### Non-Viral Transfection Process

Disclosed herein is a process that provides a high efficiency targeted gene integration approach. The methods can be used for genome engineering of any cell type, and can be used, for example, in applications where engineered cells are introduced into a patient.

In some embodiments, the methods provided herein can be used for installing a cancer treating construct, e.g. a CAR, for example against any of CD38, CD19, CD20, CD123, BCMA and the like into T cells. The efficiency of gene transfer can reach 40-80%. This approach, employing a targeted gene integration, can be used for both autologous and allogenic approaches, and importantly, does not carry a risk of secondary and unwanted cell transformation when engineered cells are introduced into a patient and is therefore safer than current conventional approaches. Additional advantages include a modified guide strand, reliable gene integration, integration of large genes, gene integration of a CAR, and gene integration of a CAR with high expression.

The examples disclose making CAR-T cells via RNA-guided endonuclease-mediated genome editing that uses phosphorothioate and 2′ O-methyl modified single-stranded or double-stranded donor DNA synthesized by PCR. Preferably, the modified single-stranded (ss) or double-stranded (ds) DNA is produced by adding three PS bonds to the nucleotides within 10 nucleotides or five nucleotides of the 5′-end of one primer. Without limiting the invention to any particular mechanism, it is believed the PS modification inhibits exonuclease degradation of the modified strand of the donor DNA. Nucleotides within ten or within five nucleotides of the 5′ end of the primer were also modified with 2′ O-methyl to avoid the non-specific binding which is caused by phosphorothioate bonds. The phosphorothioate and 2′ O-methyl modified ds donor DNA and ss donor DNA can be made through PCR, asymmetric PCR or reverse transcription. In the alternative, the final ds DNA product of a synthesis can be modified with phosphorothioate and 2′ O-methyl and dsDNA can be produced with modification on one strand only. There is further disclosed a donor DNA construct, such as a donor DNA construct having chemical modifications such as phosphorothioate and 2′ O-methyl that include a CAR construct, i.e., are designed for inserting a CAR (chimeric antigen receptor) into a defined genomic site of a host cell. Further, the present disclosure provides a host cell transfected with a CAR that lacks viral vectors that can present a safety concern.

This process—using a donor DNA with modifications on one strand—can increase knock-in efficiency at least two-fold, which is comparable with viral vector methods and has advantages for site specificity of integration and very stable for CAR expression in T cells compared to conventional retrovirus or lentivirus approaches. At least double modification of one donor chain with phosphorothioate and/or 2′ O-methyl can increase knock-in efficiency. This one step knock-out/knock-in method provides a faster and cheaper CAR-T production process for multiple cancer therapy. The ability to use double stranded DNA and avoid nuclease treatment of the donor construct and recovery of the single strand which is laborious and reduces yields is another benefit of the method.

In this application, we present a simple and robust method for knock in long dsDNA or ssDNA (e.g. ˜3 kb Anti-CD38 CAR and CD19 CAR) by modified dsDNA or ssDNA donor with phosphorothioate and 2′ O-methyl modification. We show that modified long dsDNA and ssDNA sequences are highly efficient HDR templates for the integration of CAR into primary T cells. Further we demonstrate that this method has advantages for site specificity of integration and very stable for CAR expression in T cells compared to conventional retrovirus or lenti-virus approaches.

The present disclosure provides methods for expressing a CAR gene in cell, method comprising introducing into the primary cell:

(a) a single guide RNA (sgRNA) comprising a first nucleotide sequence that is complementary to the selected knockout nucleic acid and a second nucleotide sequence that interacts with a CRISPR-associated protein (Cas) polypeptide, wherein one or more of the nucleotides of the sgRNA sequence are optionally modified nucleotides; and
(b) a Cas polypeptide, an mRNA encoding a Cas polypeptide, and/or a recombinant expression vector comprising a nucleotide sequence encoding a Cas polypeptide, or Cas polypeptide wherein the modified sgRNA guides the Cas polypeptide to the site of knockout nucleic acid, and (c) a donor target DNA comprising a 5′ HA sequences, a promoter sequence, a CAR construct, and 3′HA sequence, wherein the donor target DNA is preferably double-stranded and has both or preferably one strand modified with at least one phosphothioate bond within five nucleotides of the 5′-end of the donor for reducing; 5′ exonuclease cleavage, and optionally includes one, two three, or four 2′-O-methyl-modified nucleotides within 5 nucleotides of the 5′ end. Preferably the opposite strand to the modified strand has a 5′ terminal phosphate.

The present disclosure provides a method for inducing gene expression of a CAR gene in a primary cell, the method comprising introducing into the primary cell:

(a) a tracrRNA and a crRNA comprising a first nucleotide sequence that is complementary to the selected target knockout nucleic acid, wherein one or more of the nucleotides in the tracrRNA and a crRNA are optionally modified nucleotides; and
(b) a Cas polypeptide, an mRNA encoding a Cas polypeptide, and/or a recombinant expression vector comprising a nucleotide sequence encoding a Cas polypeptide, or a Cas polypeptide; wherein the crRNA guides the Cas polypeptide to the site of knockout nucleic acid; and (c) a donor target DNA comprising a 5′ HA sequences, a promoter sequence, a CAR construct, and 3′HA sequence, wherein the donor target DNA is preferably double-stranded and has both or preferably one strand modified with at least one phosphothioate bond within five nucleotides of the 5′-end of the donor for reducing 5′ exonuclease cleavage, and optionally includes one, two three, or four 2′-O-methyl-modified nucleotides within 5 nucleotides of the 5′ end. Preferably the opposite strand to the modified strand has a 5′ terminal phosphate.

### EXAMPLES

The examples show the advantages of the disclosed process to provide high transfection efficiency without the use of viral vectors for knocking in donor DNA and knocking out a targeted endogenous gene such as a T cell receptor (TCR) or PD-1 gene.

Buffy coats from healthy volunteer donors were obtained from the San Diego blood bank. Some fresh whole blood or leukapheresis products were obtained from StemCell Technologies. Peripheral blood mononuclear cells (PBMCs) were isolated by density gradient centrifugation. PBMCs were activated with CD3 antibody (BioLegend, San Diego, Calif.) 100 ng/mL for two days in AIM-V medium (ThermoFisher Scientific, Waltham, Mass.) supplemented with 5% fetal bovine serum (Sigma, St. Louis, Mo.) with 300 U/mL IL-2 (Proleukin) at a density of 106 cells per mL. The medium was changed every two to three days, and cells were re-plated at 10⁶ per mL. This treatment selectively amplifies T cells in the culture. In some experiments, cells were cultured in CTS™ OpTmizer™ T Cell Expansion SFM (ThermoFisher) supplemented with 5% CTS™ Immune Cell SR (Thermofisher scientific) with 300 U/mL IL-2 (Proleukin) at a density of 10\hat{ }6 cells per mL. In some experiments T cells were isolated from PBMCs using magnetic negative selection using EasySep™ Human T Cell Isolation Kit or CD3 positive selective kit (Stemcell Technology Inc.) according to the manufacturer's instructions.

For use in cytotoxicity assays, RPMI-8226 multiple myeloma cell line) cells, which express CD38, were transduced to express green fluorescent protein (GFP). K562 (human immortalized myelogenous leukemia) cells, which do not express CD38, were transduced to express R-phycoerythrin (RPE). Both cell lines were cultured in RPMI1640 medium (ATCC) supplemented with 10% fetal bovine serum (Sigma). CAR plasmids were generated with an In-Fusion® HD Cloning Kit (Takara Bio USA, Inc, Mountain View, Calif.). Backbone plasmid pAAV-MCS was purchased from Cell Biolabs (San Diego, Calif.).

In some experiments, retrovirus-transduced T cells were compared with cas-mediated knock-in cells. Transduction of T cells with the retroviral construct was performed essentially as described in Ma et al., 2004 The Prostate 61:12-25; and Ma et al., The Prostate 74 (3):286-296, 2014 (the disclosures of which are incorporated by reference herein in their entireties). In brief, the anti-CD38 CAR MFG retroviral vector plasmid DNA was transfected into Phoenix-Eco cell line (ATCC) using FuGene reagent (Promega, Madison, Wis.) to produce Ecotropic retrovirus, then harvested transient viral supernatant (Ecotropic virus) was used to transduce PG13 packaging cells with Gal-V envelope to produce retrovirus to infect human cells. Viral supernatant from PG13 cells was then used to transduce activated T cells (or PBMCs) two to three days after CD3 or CD3/CD28 activation. Activated human T cells were prepared by activating normal healthy donor peripheral blood mononuclear cells (PBMC) with 100 ng/ml mouse anti-human CD3 antibody OKT3 (Orth Biotech, Raritan, N.J.) or anti-CD3,anti-CD28 TransAct (Miltenly Biotech, German) as manufacturer's manual and 300-1000 U/ml IL2 in AIM-V growth medium (GIBCO-Thermo Fisher scientific, Waltham, Mass.) supplemented with 5% FBS for two days. 5×10⁶ activated human T cells were transduced in a 10 μg/ml retronectin (Takara Bio USA) pre-coated 6-well plate with 3 ml viral supernatant and were centrifuged at 1000 g for 1 hour at 32° C. After transduction, the transduced T cells were expanded in AIM-V growth medium supplemented with 5% FBS and 300-1000 U/ml IL2.

TABLE 1
Primers used for generating double-stranded donor DNAs:
an asterisk indicates a phosphorothioate (PS) linkage; Am, 2′-O-methylated
deoxyadenosine; Cm, 2′-O-methylated deoxycytosine; Gm, 2′-O-methylated deoxyguanosine

| Primer | Sequence | SEQ ID NO |
|---|---|---|
| Forward primer for generating anti-CD38 | 5′-T*Gm*Gm*AmGCTAGGGCACCATATT-3′ | 8 |
| donor DNA having 660 and 650 nt HAs | | |
| from TRAC gene exon 1 | | |
| Reverse primer for generating anti-CD38 | p-5′-CAACTTGGAGAAGGGGCTT-3′ | 9 |
| donor DNA having 660 and 650 nt HAs | | |
| from TRAC gene exon 1 | | |
| Forward primer for generating anti-CD38 | 5′-C*Cm*Am*TGmCCTGCCTTTACTCTG-3′ | 14 |
| donor DNA having 375 and 321 nt HAs | | |
| from TRAC gene exon 1 | | |
| Reverse primer for generating anti-CD38 | p-5′-TCCTGAAGCAAGGAAACAGC-3′ | 15 |
| donor DNA having 375 and 321 nt HAs | | |
| from TRAC gene exon 1 | | |
| Forward primer for generating anti-CD38 | 5′-A*TCm*Am*CmGAGCAGCTGGTTTCT-3′ | 18 |
| donor DNA having 171 and 161 nt HAs | | |
| from TRAC gene exon 1 | | |
| Reverse primer for generating anti-CD38 | p-5′-GACCTCATGTCTAGCACAGTTTTG-3′ | 19 |
| donor DNA having 171 and 161 nt HAs | | |
| from TRAC gene exon 1 | | |
| Forward primer for generating anti-CD38 | 5′-ATCACGAGCAGCTGGTTTCT-3′ | 20 |
| donor DNA having 171 and 161 nt HAs | | |
| from TRAC gene exon 1-unmodified | | |
| Reverse primer for generating anti-CD38 | 5′-GACCTCATGTCTAGCACAGTTTTG-3′ | 21 |
| donor DNA having 171 and 161 nt HAs | | |
| from TRAC gene exon 1-unmodified | | |
| Forward primer for generating anti-CD38 | 5′-T*Am*T*GmCmACAGAAGCTGCAAGG-3′ | 28 |
| donor DNA having 183 and 140 nt HAs | | |
| from TRAC gene exon 3 | | |
| Reverse primer for generating anti-CD38 | p-5′-TTAGGATGCACCCAGAGACC-3′ | 29 |
| donor DNA having 183 and 140 nt HAs | | |
| from TRAC gene exon 3 | | |
| Forward primer for generating anti-CD38 | p-5′-CTCCCCATCTCCTCTGTCTC-3′ | 34 |
| donor DNA having 326 and 380 nt HAs | | |
| from PD-1 locus | | |
| Reverse primer for generating anti-CD38 | 5′-Cm*Cm*T*GmACCCGTCATTCTACAG-3′ | 35 |
| donor DNA having 326 and 380 nt HAs | | |
| from PD-1 locus | | |
| Forward primer for generating anti-CD38 | 5′-TGGAGCTAGGGCACCATATT-3′ | 36 |
| donor DNA having 660 and 650 nt HAs | | |
| from TRAC gene exon 1-unmodified | | |
| Forward primer for generating anti-CD38 | 5′-ATCACGAGCAGCTGGTTTCT-3′ | 37 |
| donor DNA having 171 and 161 nt HAs | | |
| from TRAC gene exon 1 | | |

### Example 1. Simultaneous Knockout of the T-Cell Receptor Gene and Knock-In of anti-CD38 CAR in Human T Cells

In this example, the T cell receptor alpha constant (TRAC) gene was targeted with an anti-CD38 CAR construct as the donor DNA. The pAAV-TRAC-anti-CD38 construct was designed with approximately 1.3 kb of genomic DNA sequence of the T cell receptor alpha constant (TRAC) that flanks the target sequence (CAGGGTTCTGGATATCTGT (SEQ ID NO:1)) in the genome. The target sequence was identified as a site upstream of the Cas9 PAM in exon 1 of the TRAC gene for Cas9-mediated gene disruption and insertion of the donor construct. The anti-CD38 CAR gene construct (SEQ ID NO:2) comprised a sequence encoding a single chain variable fragment (scFv) specific for human CD38, followed by CD8 and CD28 hinge-CD28 transmembrane-CD28 intracellular regions and a CD3 zeta intracellular domain. An exogenous JeT promoter (U.S. Pat. No. 6,555,6674; SEQ ID NO:3) was used to initiate transcription of the anti-CD38 CAR.

To construct the pAAV-anti-CD38A2 donor plasmid which was used as a PCR template for generating donor fragments for genome editing, the anti-CD38A2 CAR construct with 650-660 bp homology arms (SEQ ID NO:4) was synthesized by Integrated DNA Technologies (IDT, Coralville, Iowa). An in-fusion cloning reaction was performed at room temperature, containing pAAV-MCS vector double digested with MluI and BstEII (50 ng), the anti-CD38A2 CAR fragment with flanking homology arms (SEQ ID NO:4) (50 ng), 1 ul 5× In-Fusion HD Enzyme Premix (Takara Bio), and nuclease-free water. The reaction was briefly vortexed and centrifuged prior to incubation at 50° C. for 30 min. Stellar™ Competent Cells (Takara Bio USA) were then transformed with the in-fusion product and plated on ampicillin-treated agar plates. Multiple colonies were chosen for Sanger sequencing (Genewiz, South Plainfield, N.J.) to identify the correct clones using the primers CTTAGGCTGGGCATTAGCAG (SEQ ID NO:5), CATGGAATGGTCATGGGTCT (SEQ ID NO:6), and GGCTACGTATTCGGTTCAGG (SEQ ID NO:7). Correct clones were cultured and the DNA plasmids from these clones were purified.

For RNA guide-directed targeting of the TCR alpha (TRAC) gene, the tracr RNA (ALT-R® CRISPR-Cas9 tracrRNA) and crispr RNA (ALT-R® CRISPR-Cas9 crRNA) were purchased from IDT (Coralville, Iowa), where the crRNA was designed to include the target sequence CAGGGTTCTGGATATCTGT (SEQ ID NO:1) that occurs directly upstream of a cas9 PAM sequence (NGG) in first exon of the TRAC gene.

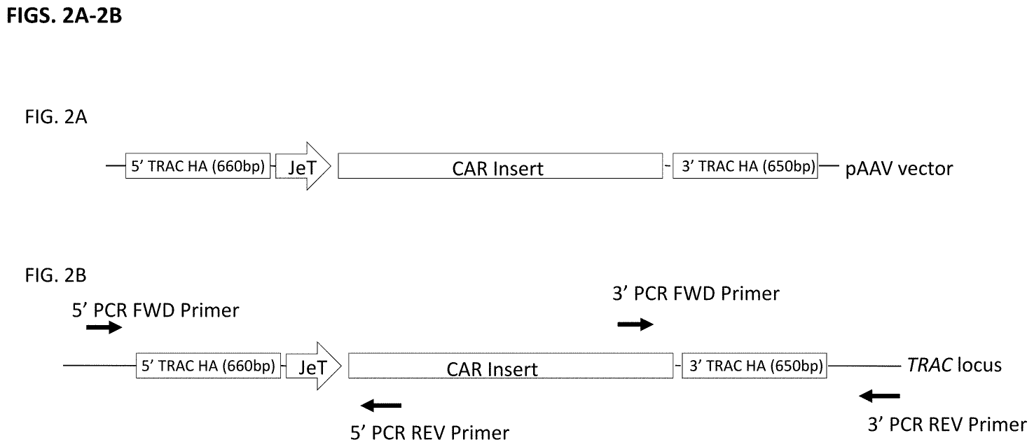

To make donor fragment DNA, PrimeSTAR Max Premix (Takara Bio USA) was used for PCR reactions. The AAV donor plasmid pAAV-anti-CD38A2 described above was used as a template. To generate a donor fragment with homology arms of 660 nt and 650 nt, the forward primer had the sequence: TGGAGCTAGGGCACCATATT (SEQ ID NO:36), and the reverse primer had the sequence: CAACTTGGAGAAGGGGCTTA (SEQ ID NO:9). In various experiments to test the effectiveness of different homology arm lengths, primers having sequences hybridizing to specific positions within the homology arms of the pAAV-anti-CD38A2 construct were used to produce donor fragments with homology arms of desired lengths by PCR. Phosphorothioate bonds (FIG. 2A) were introduced into the terminal three nucleotides at the 5′-end of the forward primer (SEQ ID NO:36) to inhibit exonuclease degradation (that is, between the first and second, second and third, and third and fourth nucleotides from the 5′ terminus). The nucleotides at the second, third and fourth positions from the 5′-end of the forward oligonucleotide primer were also 2′-O-methyl modified to avoid non-specific binding, potentially caused by the phosphorothioate (PS) backbone of the terminal 3 nucleotides (SEQ ID NO:8, FIG. 2B). The reverse primer (SEQ ID NO:9) was modified by 5′-end phosphorylation so that the strand could be digested by a strandase provided by the Guide-it™ Long ssDNA Production System kit (Takara Bio USA). To produce the donor DNA fragment, the thermocycler settings were: one cycle of 98° C. for 30 s, 35 cycles of 98° C. for 10 s, 66° C. for 5 s, 72° C. for 30s and one cycle of 72° C. for 10 min. Digestion with the strandase was done according to the manufacturer's instructions (Takara Bio USA), and ssDNA was purified using the NucleoSpin Gel and PCR Clean-Up kits (Takara Bio USA). The concentration of ssDNA was determined by NanoDrop (Denovix, Wilmington, Del.). As controls, donor fragments were produced with unmodified primers, such that the resulting donor fragment had no chemical modifications (no PS or 2′-O-methyl groups) or had the PS modification only (no 2′-O-methyl groups).

To generate TCR knockouts/anti-CD38 CAR knock-ins, T cells were activated by adding CD3 to the cultures. About 48 to 72 hours after initiating T-cell activation with CD3, the PBMC cultures including activated T cells were electroporated with SpCas9 protein plus crRNA (containing guide sequence SEQ ID NO:1) and tracrRNA using a Neon® Transfection System (ThermoFisher Scientific) and 10-μl tip or 100-μl tips. Briefly, Alt-R CRISPR-Cas9 crRNA and Alt-R tracrRNA (IDT) were first mixed and heated at 95° C. for 5 min. The mixture was then removed from heat and allow to cool to room temperature (15-25° C.) on the bench top for about 20 min. For each transfection, 10 μg SpCas9 protein (IDT) was mixed with 200 pmol crRNA:tracrRNA duplex to form RNPs. 1×10⁶ cells were mixed with the RNP and electroporated with 1700 V, 20 ms pulse width, 1 pulse. One to two hours later, 10 ug single-stranded donor DNA was electroporated into the cells with 1600 V, 20 ms pulse width, 1 pulse. In some cases, T cells were mixed with the RNP and donor DNA and RNP and donor were electroporated at the same time. Following electroporation cells were diluted into culture medium and incubated at 37° C., 5% CO₂.

As controls for the cas-mediated knock-in methods, CAR-expressing PBMCs were generated by transduction of T cells with a retrovirus that included the same anti-CD38A2 expression cassette (SEQ ID NO:2) in the retroviral vector that was used to make the donor fragment employed in CRISPR targeting.

To determine knock-in efficiency by detecting CAR expression of transformed cells by FACS, transfected or transduced PBMCs were washed with DPBS/5% human serum albumin, then stained with anti-CD3-BV421 antibody SK7 (BioLegend) and PE conjugated anti-CD38-Fc protein (Chimerigen Laboratories, Allston, Mass.) for 30-60 min at 4° C. CD3 and anti-CD38 CAR expression were analyzed using iQue Screener Plus (Intellicyte Co.) Negative controls were cells that had been transfected with an RNP that included cas9 protein complexed with a hybridized tracrRNA and crRNA targeting the first exon of the TRAC gene, but were not transfected with the anti-CD38 CAR donor DNA. PBMCs that had been transfected with the RNP that included the guide targeting the TRAC locus were subsequently transduced with a retrovirus that included the anti-CD38 CAR construct as described above and analyzed for expression of the anti-CD38 CAR as well. FIG. 3A shows that 8 days after transfection no expression of an anti-CD38 construct was detected in cells transformed with the RNP (for knocking out the TRAC gene) in the absence of a donor fragment for expression of the anti-CD38 CAR (leftmost panel). On the other hand, PBMCs that had a TRAC knockout and were subsequently transduced with a retrovirus that included a construct for expressing the anti-CD38 CAR did show expression of the anti-CD38 CAR in about 70% of the cells 8 days after transfection (rightmost panel of FIG. 3A). For cultures transformed with anti-CD38 CAR ss donor DNA in addition to an RNP targeting exon 1 of the TRAC gene, approximately 12% of the population that received the ss donor DNA having no chemical modifications and approximately 13% of cultures that were transduced with ss donor DNA having only PS backbone modifications on nucleotides near the 5′-end of the donor DNA (introduced by using a PCR primer having PS bonds between nucleotides 1 and 2, 2 and 3, and 3 and 4, numbering from the 5′ end) demonstrated expression of the anti-CD38 construct. Adding methyl groups to the 2′ oxygen of the three nucleotides at the second, third, and fourth nucleotides from the 5′-end of the donor fragment strand that also included PS modifications (by using the primer of SEQ ID NO:8 that included these modifications to generate the donor DNA by PCR) resulted in significantly higher expression of the anti-CD38 CAR in the transfected population, where expression of the anti-CD38 CAR was seen in approximately 20% of the cells that received the ‘double modified’ (2′-O-methyl and PS) single-stranded donor fragment at 8 days. Notably, chemical modifications of the donor DNA did not affect viability of the transfected cultures.

Increased expression of the anti-CD38 CAR was observed over time in cultures that had been transfected with anti-CD38 CAR donor fragments plus RNPs targeting the TRAC gene. At 10 days post-transfection, flow cytometry of PBMC cultures transfected with unmodified single-stranded donor or single-stranded donor modified to include PS linkages on the 5′-most three nucleotides demonstrated that among all cultures that were transfected with the TRAC-targeting RNP, at least 80% of the cells did not express the TCR. Moreover, in cultures transfected with the anti-CD38 CAR donor in addition to the TRAC-targeting RNP, at least 42% of the cells that did not express the TCR expressed the anti-CD38 construct (FIG. 3B, panels 2-4). For cultures transfected with an anti-CD38 CAR donor fragment with both PS and 2′-O-methyl groups on 5′-proximal nucleotides, 57% of the cells were expressing the anti-CD38 construct by day ten. At the same time, the expression of the anti-CD38 CAR in cultures that had been transduced with the retrovirus dropped to about half of what had been seen at 8 days, to approximately 34% of the cells on day ten post-transfection or transduction. Analysis of the culture transfected with doubly modified ss donor and the retrovirus-transduced culture at day 20 (FIG. 3C) showed that expression of the anti-CD38 construct in the cultures had stabilized, with the cas9-modified culture that had been transfected with a ss donor having both PS and 2′-O-methyl modifications at the 5′ end demonstrating 54% of the TCR-negative cells were expressing the construct and the culture that had been transduced with a retrovirus demonstrating 31% of the TCR-negative cells were expressing the construct.

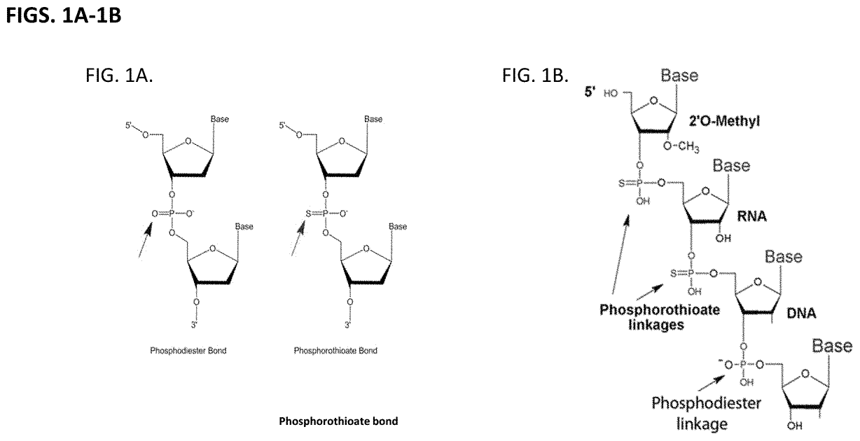

To confirm the occurrence of homology directed repair (HDR) at the targeted locus in Exon 1 of the TRAC gene, PCR was performed on DNA isolated from cultures to verify that the donor fragment had inserted into the TRAC site targeted by the guide RNA. Genomic DNA was amplified from non-transfected activated T cells (ATCs), TRAC knockout cells that were transformed with the RNP that included the TRAC Exon 1 guide RNA, and from T cells transfected with the RNP plus phosphorothioate and 2′ O-Methyl modified donor DNA to detect targeted insertion of an anti-CD38 CAR transgene into the TRAC locus. To confirm the position of the donor DNA in the genome, oligonucleotide primers were targeted to sequences outside of the TRAC homology arms but adjacent to the homology arm sequences in the genome. A total of 1×10⁵ cells were resuspended in 30 μL of Quick Extraction solution (Epicenter) to extract the genomic DNA. The cell lysate was incubated at 65° C. for 5 min and then at 95° C. for 2 min and stored at −20° C. The concentration of genomic DNA was determined by NanoDrop (Denovix). Genomic regions containing the TRAC target sites were PCR-amplified using the following primer sets: 5′ PCR forward primer on TRAC: CTGCTTTCTGAGGGTGAAG (SEQ ID NO:10), 5′ PCR Reverse primer on CAR: CTTTCGACCAACTGGACCTG (SEQ ID NO:11); 3′ Forward primer on CAR: CGTTCTGGGTACTCGTGGTT (SEQ ID NO:12), 3′ Reverse primer on TRAC: GAGAGCCCTTCCCTGACTTT (SEQ ID NO:13) (see FIG. 1B). Both primer sets were designed to avoid amplifying the HDR templates by annealing outside of the homology arms.

The concentration of genomic DNA was determined by NanoDrop (Denovix). Both primer sets were designed such that one primer of the pair annealed to a site in the genome outside of the homology arm, and the other primer of the pair annealed to a site within the coding region of the construct (i.e., not in a homology arm). The PCR contained 400 ng of genomic DNA and Q5 high fidelity 2× mix (New England Biolabs). The thermocycler setting consisted of one cycle of 98° C. for 2 min, 35 cycles of 98° C. for 10 s, 65° C. for 15 s, 72° C. for 45 s and one cycle of 72° C. for 10 min. The PCR products were purified on 1% agarose gel containing SYBR Safe (Life Technologies). The PCR products were then eluted from the agarose gel and isolated using NucleoSpin® Gel and PCR Clean-up kit (MACHEREY-NAGEL GmbH & Co. KG). The PCR products were submitted for Sanger sequencing (Genewiz). FIG. 4 provides a photograph of the gel separating PCR products. The positive bands corresponding to the anti-CD38 construct adjacent to genomic sequences adjacent to the homology arms in the genome at the 5′ and 3′ ends of the construct were only seen in cells transfected with donor DNA (lanes 3 and 6) and not in non-transfected ATCs (lanes 1 and 4) or TRAC knock out cells (lanes 2 and 5). Sequencing of these PCR products confirmed that they included the anti-CD38 CAR sequence. SEQ ID NO:

To test for function of transfected cells, three weeks after electroporation, the activated T cells that had been transfected with the anti-CD38 CAR targeted to the TRAC locus were starved with IL-2 overnight and tested in specific killing assays (FIG. 5). The activated T cells were co-cultured with a target cell mixture of CD38 positive RPMI-8226/GFP cells and CD38 negative K562/RPE cells. The incubation effector-to-target cell ratio ranged from 10:1 to 0.08:1. After overnight incubation, the cells were analyzed by flow cytometry to measure the GFP-positive and RPE-positive cell populations to determine the specific target cell killing by anti-CD38A2 CART cells. FIG. 5 shows that while non-transfected ATC cells showed some toxicity at the highest effector to target ratios, TRAC knockout cells showed virtually no killing regardless of effector-to-target cell ratio. The anti-CD38A2 CART cells however exhibited potent killing activity of CD38 positive cells—RPMI8226 but not CD38 negative cells—K562 (FIG. 5). T cells that had integrated the chemically modified donor that included the anti-CD38 CAR cassette demonstrated cytotoxicity toward target cells similarly to that of cells transduced with retrovirus that included the anti-CD38 CAR construct.

The transfected activated T cells (ATCs) were also tested for cytokine secretion (FIG. 6). T cells were starved in IL-2 free medium overnight. Anti-CD38 CAR-T cells or ATC controls were then co-cultured with CD38 negative K562 or CD38 positive RPMI8226 cells. The incubation effector to target cell ratio was 2:1. After overnight incubation, the cells were centrifuged to collect the supernatants for quantitating cytokine IL-2, IFN-gamma and TNF alpha (Affymetrix eBioscience) according to the manufacturer's instructions. The gene-edited TCR knockout anti-CD38A2 CART cells also released similar amount of IFN-y and other pro-inflammatory cytokines when co-cultured with CD38 positive tumor cells (RPMI8226) but not CD38 negative cells (K562).

In summary, in vitro cellular functional studies did not reveal any notable differences between TRAC-site-specific integrated anti-CD38A2 CAR achieved by this novel and efficient process and virus-mediated randomly integrated anti-CD38A2 CAR, in terms of both specific killing assay (FIG. 5) and cytokine secretion assay (FIG. 6).

### Example 2. Reducing Length of Homology Arms of Donor DNAs

When synthesizing donor DNA by PCR, the nuclease reaction and resulting purification of the single stranded donor fragment is time consuming, typically results in losses in the yield of donor fragment for transfections, and can be difficult to control the length of homology arms (homology can be over-chewed). In further experiments testing the efficiency of directed gene knockouts and antibody construct knock-ins, double-stranded donor DNAs were tested to eliminate the nuclease digestion of the PCR-synthesized donor.

For knock-in of the anti-CD38 CAR construct, donor fragments having homology arms (HAs) of different lengths were produced. The pAAV-TRAC-anti-CD38 construct described in Example 1 that included the anti-CD38 cassette plus TRAC exon 1 homology arms of 660 and 650 nts (SEQ ID NO:4) was used as the template. A first set of primers, SEQ ID NO:8 and SEQ ID NO:9, was used to generate a donor fragment having homology arms of 660 nt and 650 nt from this template. A second set of primers, SEQ ID NO:14 and SEQ ID NO:15, was used to generate a donor fragment having homology arms of approximately 350 nt (375 and 321 nucleotides), where the primer of SEQ ID NO:14 had PS linkages between the between first and second, second and third, and third and fourth nucleotides from the 5′ terminus and had 2′-O-methyl-modified nucleotides at positions 2, 3, and 5. A third set of primers, SEQ ID NO:18 and SEQ ID NO:19, was used to generate a donor fragment having homology arms of approximately 165 nt (171 and 161 nts), where the primer of SEQ ID NO:18 had PS linkages between the between first and second, third and fourth, and fourth and fifth nucleosides from the 5′ terminus and had 2′-O-methyl-modified nucleotides at positions 3, 4, and 5. In each case, the forward primer (SEQ ID Nos: 8, 14, and 18) was designed to have three PS linkages within the 5′terminal-most five nucleotides (for example, between any of the first and second, second and third, third and fourth, and fourth and fifth nucleosides from the 5′ terminus of the primer, and three 2′-O-methyl groups occurring in any of the five 5′terminal-most nucleotides. In each case, the reverse primer (SEQ ID Nos: 9, 15, and 17) had a 5′ terminal phosphate (see Table 1).

Each of the primer sets was used to generate a donor DNA molecule having multiple PS and 2′-O methyl modifications proximal to the 5′end of one strand of the donor and a 5′ phosphate at the 5′ terminus of the opposite strand of the donor. RNPs were assembled to include tracr and crRNAs as described in Example 1, where the crRNA included the target sequence of SEQ ID NO:1, a sequence found in exon 1 of the TRAC gene. The donor molecules, having homology arms of approximately 665, 350, and 165 base pairs in length, were independently transfected into activated T cells as described in Example 1 except that donor fragments and RNPs were transfected in the same electroporation under conditions for electroporating the RNP (using a Neon® Transfection System (ThermoFisher Scientific) 1700 V, 20 ms pulse width, 1 pulse). As a control, activated T cells were transfected with the RNP in the absence of a donor fragment, which should result in knockout of the targeted TRAC locus, but without donor DNA insertion. To test for expression of the T cell receptor and the anti-CD38 CAR construct, flow cytometry was performed as provided in Example 1. FIG. 7 shows that, as expected, the T cell culture transfected with the RNP only had low levels of expression of the T cell receptor and also demonstrates no expression of the anti-CD38 CAR. T cells transfected with the RNP plus donor DNAs having homology arms of different sizes however show low levels of T cell receptor expression and good expression of anti-CD38 CAR in the cultures, demonstrating that transfection of a double-stranded DNA in highly effective for targeted knock-ins. Further, the shortest HA lengths tested, 161/171 nt, worked at least as well as longer lengths, with the percentages of knockout cells expressing the introduced construct being approximately 24% for approximately 665 nt arms, approximately 30% for approximately 350 nt arms, and approximately 38% for approximately 165 nt arms. The short homology arms are thus found to be very effective in targeted knock in genome modification using double-stranded DNA donors, which has the benefit of allowing for smaller constructs and/or allowing for more capacity in a construct to allow inclusion of additional or lengthier sequences to be included in the donor DNA.

### Example 3. Modified Versus Non-Modified Double-Stranded Donor DNA

Donor DNAs that included anti-CD38 CAR and having the approximately 165 nt TRAC exon 1 homology arms as set forth in Example 2, above, were synthesized using primers with and without nucleotide modifications to test their relative effectiveness in promoting HDR. In the first case, primer SEQ ID NO:18 had three PS linkages, occurring between first and second, third and fourth, and fourth and fifth nucleosides and three 2′-O-methyl-modified nucleotides within the first five nucleotides of the 5′ terminus of the primer (at nucleotide positions 2, 3, and 5) and primer SEQ ID NO:19 had a 5′ terminal phosphate (Table 1). These primers were used to generate a donor DNA with the corresponding nucleotide modifications (i.e., three PS linkages and three 2′-O-methyl groups within five nucleotides of the 5′ terminus of the first strand of the donor DNA product, and a phosphate on the 5′ end of the second strand of the donor DNA product). In the second case, primer SEQ ID NO:37 was identical to primer SEQ ID NO:18 except that primer SEQ ID NO:37 lacked chemical modifications see Table 1). The SEQ ID NO:37 primer and the SEQ ID NO:19 primer lacking a 5′ terminal phosphate were used to generate a donor DNA with no nucleotide modifications having the anti-CD38 CAR cassette. These donor DNAs were transfected as double-stranded DNA molecules (with no denaturation or nuclease digestion of either strand) along with RNPs that included a tracr RNA and a crRNA that included the target sequence of SEQ ID NO:1 (within exon 1 of the TRAC gene) into activated T cells. In the electroporations with double stranded DNA as donor, 5 ug dsDNA was used to transfect one million activated T cells.

As in Example 2, control activated T cells were transfected with the RNP in the absence of a donor fragment, which should result in knockout of the targeted TRAC locus without construct insertion. To test for expression of the T cell receptor and the anti-CD3 CAR construct, flow cytometry was performed essentially as provided in Example 1. The results, shown in FIG. 8, show that transfection with the RNP and a modified double stranded donor resulted in at least twice the expression of the anti-CD38 construct across the culture as compared with transfection with the RNP and the unmodified double-stranded donor, resulting in over 50% of the cells of the culture expressing the anti-CD38 CAR transgene and not expressing the TCR (CD3 negative).

Sequencing of PCR products produced using primers to diagnose the insertion locus (see FIG. 2B) provided sequences demonstrating the anti-CD38 CAR donor fragment integrated into exon 1 of the TRAC gene. The PCR product sequences (SEQ ID NO:39 and SEQ ID NO:40) included sequences adjacent to the homology arm in the genome, the homology arm present in the donor fragment, and portions of the anti-CD38 CAR in a single PCR product, demonstrating the expected insertion.

### Example 4. HDR-Mediated Knock-In of Anti-CD19 and Anti-BCMA CAR Constructs with Simultaneous TCR Knockout

Additional donor DNAs that included anti-CD19 CAR and anti-BCMA CAR expression constructs were also tested for insertion into the TRAC locus.

An anti-CD19 CAR construct that included an anti-CD19 CAR cassette (SEQ ID NO:22) that included the Jet promoter (SEQ ID NO:3), and intron, an anti-CD19 CAR construct, and an SV40 polyA sequence was made essentially as described for the anti-CD38 CAR pAAV construct described in Example 1 and was cloned in a vector flanked by the TRAC gene exon 1 homology arms (HAs) of SEQ ID NO:20 and SEQ ID NO:21. The anti-CD19 CAR with HAs pAAV construct was used as a template in PCR reactions as provided in Example 1 using the primers provided as SEQ ID NO:18 and SEQ ID NO:19 that result in the production of modified donor DNA having HAs of approximately 170 and 160 nucleotides (see Table 1). The forward primer (SEQ ID NO:18) had three PS bonds between the first and second, third and fourth, and fourth and fifth nucleosides and three 2′-O-methyl modifications at nucleotides 3, 4, and 5 when numbering from the 5′-terminus of the primer. The reverse primer (SEQ ID NO:19) had a 5′-terminal phosphate. The resulting double-stranded donor DNA was therefore synthesized to have the corresponding modifications, a first strand with three PS and three 2′-O-methyl modifications within five nucleotides of the 5′-terminus, and a second strand with a 5′-terminal phosphate.

The double-stranded chemically modified donor fragment having the sequence of SEQ ID NO:38 with the nucleotide modifications of primers SEQ ID NO:18 and SEQ ID NO:19 described above incorporated was used to transfect cells along with an RNP that was produced according to the methods provided in Example 1, where the crRNA of the RNP included the target sequence of SEQ ID NO:1, targeting exon 1 of the TRAC gene. As a control, activated T cells were transfected with the RNP in the absence of a donor fragment, which should result in knockout of the targeted TRAC locus without construct insertion. Flow cytometry was performed essentially as described in Example 1 to evaluate the efficiency of introducing a different construct into the TRAC locus, except that anti-CD19 CAR expression was detected by CD19-Fc (Speed Biosystem) followed by APC anti-human IgG Fcγ (Jackson Immunoresearch). The results are shown in FIG. 9, where it can be seen that the anti-CD19 CAR was expressed in the absence of T cell receptor expression in approximately 42% of the cells in the culture.

An anti-BCMA CAR construct was made through replacing the CD38 CAR with BCMA CAR based on the anti-CD38 CAR pAAV construct described in Example 1. The BCMA CAR fragment was synthesized by IDT. The sequence of the insert is provided as SEQ ID NO:23. The anti-BCMA CAR construct was used as a template in PCR reactions as set forth in Example 1 using the primers provided as SEQ ID NO:18 and SEQ ID NO:19 that result in the production of donor DNA having HAs of approximately 160-170 nucleotides (see Table 1). The forward primer (SEQ ID NO:18) had three PS and three 2′-O-methyl modifications within five nucleotides of the 5′-terminus of the primer. The reverse primer (SEQ ID NO:19) had a 5′-terminal phosphate. The resulting double-stranded donor DNA was therefore synthesized to have a first strand with three PS and three 2′-O-methyl modifications within five nucleotides of the 5′-terminus, and a second strand with a 5′-terminal phosphate.

The double-stranded donor fragment having the sequence of SEQ ID NO:37, having modified nucleotides by incorporation of chemically modified primers as provided above, was used to transfect cells along with an RNP that was produced according to the methods provided in Example 1, where the crRNA of the RNP included the target sequence of SEQ ID NO:1, targeting exon 1 of the TRAC gene. As a control, activated T cells were transfected with the RNP in the absence of a donor fragment, which should result in knockout of the targeted TRAC locus without construct insertion. Flow cytometry was performed as described in Example 1 to evaluate the efficiency of introducing a different construct into the TRAC locus, except that anti-BCMA CAR expression was detected by PE or APC conjugated BCMA-Fc (R&D). The results are shown in FIG. 10, where it can be seen that the anti-BCMA CAR was expressed in the absence of T cell receptor expression in approximately 66% of the cells in the culture.

### Example 5. HDR Mediated Knock-In Targeting TRAC Exon 3

To test the efficiency of inserting donor DNAs into loci other than exon 1 of the TRAC gene using the methods for donor insertion provided herein, an anti-CD38 CAR construct was made for producing a donor DNA having HAs from Exon 3 of the TRAC gene. In this case, the construct was produced essentially as described in Example 1 for the TRAC exon 1 targeting construct, except that the HAs (5′ HA SEQ ID NO:24 (183 nt) and 3′ HA SEQ ID NO:25 (140 nt)) were sequences surrounding the exon3 target site (SEQ ID NO:26). The sequence of the insert of the pAAV construct that was then produced as a donor DNA with TRAC gene exon 3 homology arms is provided as SEQ ID NO:27. To generate the donor fragment, the forward primer (SEQ ID NO:28) included PS linkages between first and second, second and third, and third and fourth nucleosides and 2′-O-methyl modifications on the second, fourth, and fifth positions from the 5′-terminus, and the reverse primer (SEQ ID NO:29) had a 5′-terminal phosphate. The resulting double-stranded donor DNA that incorporated the primers had a first strand with corresponding PS and 2′-O-methyl modifications on the 5′-terminal most nucleotides, and a second strand having a 5′-terminal phosphate.

The double-stranded donor fragment having modified nucleotides by incorporation of the primers above and having the sequence of SEQ ID NO:27 was used to transfect cells along with an RNP that was produced according to the methods provided in Example 1, where the crRNA included the target sequence of SEQ ID NO:26, targeting exon 3 of the TRAC gene. As a control, activated T cells were transfected with the RNP in the absence of a donor fragment, which should result in knockout of the targeted TRAC locus without construct insertion. A further control was non-transfected activated T cells (ATCs). Flow cytometry was performed essentially as described in Example 1. The results are shown in FIG. 11, where it can be seen that transfection with the RNP or the RNP plus donor DNA result in greater than 80% of cells across the culture losing TCR expression. Further, anti-CD38 CAR was expressed in the absence of T cell receptor expression in approximately 42% of the cells in the culture that was transfected with the targeting RNP plus the donor DNA with HAs derived from the TRAC gene exon 3.

Sequencing of PCR products produced using primers to diagnose the insertion locus (see FIG. 2B) provided sequences demonstrating the anti-CD38 CAR donor fragment integrated into exon 3 of the TRAC gene. The PCR product sequences (SEQ ID NO:41 and SEQ ID NO:42) included sequences adjacent to the homology arm in the genome, the homology arm present in the donor fragment, and portions of the anti-CD38 CAR in a single PCR product, demonstrating the expected insertion.

FIG. 12 compares targeting of the anti-CD19 CAR to exon 3 and exon 1 of the TRAC gene. The anti-CD19 CAR donor DNA directed to exon 3 is synthesized to include the anti-CD19 CAR cassette (SEQ ID NO:22) as set forth in the Examples above, where the anti-CD19 expression cassette is flanked by sequences from the exon 3 locus (SEQ ID NO:24 and SEQ ID NO:25) as set forth above. The anti-CD19 CAR donor directed to exon 1 (having the sequence of SEQ ID NO:38) is provided in Example 4. Each of these constructs—one having the anti-CD19 CAR cassette (SEQ ID NO:22) flanked by TRAC exon 1 HAs (SEQ ID NO:18 and SEQ ID NO:19), and the other having the anti-CD19 CAR cassette (SEQ ID NO:22) flanked by TRAC exon 3 HAs (SEQ ID NO:24 and SEQ ID NO:25), was used to produce donor fragment using modified forward primers having PS and 2′-O-methyl modifications on the three 5′-terminal most nucleotides. The reverse primers had 5′-terminal phosphates. The primers for producing the anti-CD19 CAR donor flanked by exon 1 HAs were SEQ ID NO:18 and SEQ ID NO:19, where the SEQ ID NO:18 primer included PS linkages between first and second, third and fourth, and fourth and fifth nucleosides and 2′-O methyl groups at position 3, position 4, and position 5 from the 5′ end. The primers for producing the anti-CD19 CAR donor flanked by exon 3 HAs were SEQ ID NO:28 and SEQ ID NO:29, where the SEQ ID NO:28 primer had PS linkages between the first and second, second and third, and third and fourth nucleosides from the 5′ end and 2′-O-methyl groups at position 2, position 4, and position 5 from the 5′ end. The resulting double-stranded donor DNAs thus had a first strand with corresponding PS and 2′-O-methyl modifications on the 5′-terminal end nucleotides, and a second strand having a 5′-terminal phosphate.

The donor fragments were independently transfected into activated T cells with RNPs. RNPs were produced as described in Example 1, except that for targeting TRAC gene exon 1, the target sequence of the crRNA was SEQ ID NO:1, and for targeting TRAC gene exon 3, the target sequence of the crRNA was SEQ ID NO:26. As can be seen in FIG. 12, approximately 41% of the culture that was transfected with an RNP targeting exon 3 of the TRAC gene and a donor fragment for expressing the anti-CD19 CAR were both TCR negative and positive for anti-CD19 CAR, while approximately 20% of the culture that was transfected with an RNP targeting exon 1 of the TRAC gene and a donor fragment for expressing the anti-CD19 CAR were both TCR negative and positive for anti-CD19 CAR. T cell cultures transduced with a retrovirus including the anti-CD19 CAR expression cassette demonstrated a higher percentage of anti-CD19 CAR expressing cells, but these cells did not have a TCR knockout.

### Example 6. HDR Mediated Knock-In Targeting PD-1 Gene

The PD-1 locus was also targeted with a CAR construct. In this case the anti-CD38 CAR cassette (SEQ ID NO:2) was juxtaposed with homology arms (SEQ ID NO:30 and SEQ ID NO:31) having sequences of the PD-1 locus that surround a target site (SEQ ID NO:32) using the methods essentially as described in Example 1 to provide a template for producing donor DNA.

Donor DNA was produced essentially as described in Example 1, using a forward primer (SEQ ID NO:34) that included a 5′ phosphate and a reverse primer that included phosphorothioate linkages between first and second, second and third, and third and fourth nucleosides from the 5′ end as well as 2′-O-methyl groups on the first, second, and fourth nucleosides from the 5′ end (SEQ ID NO:35), see Table 1.

The double-stranded chemically modified donor fragment (SEQ ID NO:33) was used to transfect cells along with an RNP produced according to the methods provided in Example 1, where the crRNA included the target sequence of SEQ ID NO:32, targeting the PD-1 gene. As a control, activated T cells were transfected with the RNP in the absence of a donor fragment, which generates a knockout of the targeted TRAC locus without CAR construct insertion. A further control was non-transfected activated T cells (ATCs). Flow cytometry was performed essentially as described in Example 1, where an additional control of nontransfected activated T cells (ATCs) was included. A BV421-conjugated antibody to PD-1(EH12.2H7, BioLegend) was used to detect PD-1 expression.

The results are shown in FIG. 13, where it can be seen the percentage of cells expressing PD-1 dropped from approximately 19% in ATCs to approximately 4% in the cells of cultures transfected with the RNP targeting the PD-1 locus (PD-1 RNP). The anti-CD38 CAR was expressed in the absence of T cell receptor expression in approximately 27% of the cells in the culture that was transfected with the PD-1 targeting RNP plus a donor with HAs having homology to the PD-1 locus. As a comparison, about 32% of cells of a culture transfected with an RNP targeting exon 1 of the TCR and an anti-CD38 CAR donor fragment with HAs having homology to sequences of exon 1 of the TRAC gene.

Sequencing of PCR products produced using primers to diagnose the insertion locus (see FIG. 2B) provided sequences demonstrating the anti-CD38 CAR donor fragment integrated into the PD-1 gene. The PCR product sequences (e.g., SEQ ID NO:43) included sequences adjacent to the homology arm in the genome, the homology arm present in the donor fragment, and portions of the anti-CD38 CAR in a single PCR product, demonstrating the expected insertion.

FIG. 14 provides the results of a cytotoxicity assay that was performed using PBMCs and isolated T cells from cultures transfected with the anti-CD38 CAR donor fragment and an RNP targeting the PD-1 locus (“PD-1 KOKI PBMC” and “PD-1 KOKI T cell” respectively). These modified cells showed a high level of cytotoxicity toward target cells in the assay with respect to control cells that had a PD-1 gene knockout but did not receive a CAR construct (“PD-1 KO”) and control cells that had a TRAC gene knockout but did not receive a CAR construct (“TRAC-1 KO”) and were outperformed somewhat by cells that were transfected the anti-CD38 CAR donor fragment and an RNP targeting the TRAC locus (“TRAC KOKI”), likely due to the lower efficiency of donor CAR construct integration at the PD-1 site that was observed (FIG. 13).

What is claimed is:

1. A method for site-specific integration of a donor DNA into a target DNA molecule, comprising introducing into a cell:
an RNA-guided endonuclease or a nucleic acid molecule encoding an RNA-guided endonuclease;
at least one engineered guide RNA or at least one nucleic acid molecule encoding an engineered guide RNA; and
a donor DNA molecule comprising at least two nucleic acid modifications;
wherein the guide RNA comprises a target sequence designed to hybridize with a target site in the target DNA and the donor DNA is inserted into the target DNA molecule at the target site.

2. A method according to claim 1, wherein the at least two nucleic acid modifications are on a single strand of the donor DNA molecule.

3. A method according to claim 1, wherein one or more nucleic acid modifications are a modification of one or more nucleotides or nucleotide linkages within 10 nucleotides of the 5′ end of the modified strand of the donor DNA molecule.

4. A method according to claim 1, wherein one or more nucleic acid modifications is a backbone modification.

5. A method according to claim 3, wherein one or more nucleic acid modifications is a phosphorothioate modification.

6. A method according to claim 1, wherein one or more nucleic acid modifications is a modification or substitution of a nucleobase.

7. A method according to claim 1, wherein one or more nucleic acid modifications is a modification or substitution of a sugar.

8. A method according to claim 7, wherein one or more nucleic acid modifications is a 2′-O-methyl group modification of deoxyribose.

9. A method according to claim 1, wherein the donor DNA molecule is a double stranded DNA molecule.

10. A method according to claim 1, wherein the donor DNA molecule has a 5′ terminal phosphate on the strand opposite to the modified strand.

11. A method according to claim 9, wherein the donor molecule has between one and three phosphorothioate modifications on the backbone within ten nucleotides of the 5′ terminus of one strand of the donor molecule and between one and three 2′-O-methyl nucleotide modifications within ten nucleotides of the 5′ terminus of one strand of the donor molecule.

12. A method according to claim 10, wherein the donor molecule has between one and three phosphorothiorate modifications on the backbone within five nucleotides of the 5′ terminus of one strand of the donor molecule and between one and three 2′-O-methyl nucleotide modifications within five nucleotides of the 5′ terminus of one strand of the donor molecule.

13. A method according to claim 1, wherein the donor DNA molecule includes homology arms flanking a sequence for integration into the genome.

14. A method according to claim 1, wherein the guide RNA is a crRNA.

15. A method according to claim 13, wherein the method further comprises introducing a tracr RNA into the cell.

16. A method according to claim 1, wherein the guide RNA is a chimeric guide RNA.

17. A method according to claim 1, wherein the RNA-guided endonuclease is Cas9, Cas12a, Cas12b, Cas13, Cas14, or CasX.

18. A method according to claim 1, wherein at least one guide RNA is introduced into the cell.

19. A method according to claim 1, wherein an RNA-guided endonuclease is introduced into the cell.

20. A method according to claim 19, wherein the RNA-guided endonuclease is introduced into the cell as a ribonucleoprotein.

21. A system for targeted integration of a donor DNA into a target locus, comprising,
an RNA-guided endonuclease or a nucleic acid molecule encoding an RNA guided endonuclease;
a guide RNA or a nucleic acid molecule encoding a guide RNA; and
a double-stranded donor DNA molecule, wherein the donor DNA molecule includes one or more phosphorothioate bonds on a single strand of the double stranded DNA molecule within five nucleotides of the 5′ terminus of the modified strand of the nucleic acid molecule.

22. The system of claim 1, wherein the system comprises an RNA-guided endonuclease.

23. The system of claim 1, wherein the system comprises a guide RNA.

24. The system of claim 1, wherein the donor DNA molecule further comprises at least one modification of a sugar moiety or nucleobase of the modified strand within five nucleotides of the 5′ terminus of the modified strand of the nucleic acid molecule.

25. The system of claim 1, wherein the donor DNA has homology arms flanking a sequence of interest for integration into the genome.

26. A composition for generating a donor DNA molecule comprising
a first primer having one or more phosphorothioate bonds and one or more modified nucleotides on a single strand of the double stranded DNA molecule within five nucleotides of the 5′ terminus of the modified strand of the nucleic acid molecule; and
a second primer having a 5′ terminal phosphate.

27. A composition according to claim 26, wherein the first and second primers are homologous to sequences on opposite sides of a target site for an RNA-guided endonuclease in a target genome.
